(19) United States
(12) Patent Application Publication
(10) Pub. No.: US 2024/0076725 A1
(43) Pub. Date: Mar. 7, 2024

(54) MICROFLUIDIC SUBSTRATE, MICROFLUIDIC CHIP, METHODS FOR PREPARING AND USING THE CHIP

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Zhukai Liu, Beijing (CN); Ruijun Deng, Beijing (CN); Kang Peng, Beijing (CN); Ding Ding, Beijing (CN)

(21) Appl. No.: 18/262,397

(22) PCT Filed: May 10, 2022
(86) PCT No.: PCT/CN2022/092031

§ 371 Date: Jul. 21, 2023

(30) Foreign Application Priority Data
Oct. 28, 2021 (WO) PCT/CN2021/127002

(51) Int. Cl. C12Q 1/6844; B01L 3/00; B01L 7/00

(52) U.S. Cl. CPC C12Q 1/6844; B01L 3/502715; B01L 7/52; B01L 2200/16; B01L 2300/0654; B01L 2300/0819; B01L 2300/1827

(57) ABSTRACT

The present disclosure provides a microfluidic substrate, a microfluidic chip, a method for preparing the microfluidic chip, and a method for using the microfluidic chip. The microfluidic substrate includes a substrate including a plurality of microcavity regions arranged m an array, each of the plurality of microcavity regions includes a first portion and a second portion that are stacked, and the depth of the first portion is x, and the first portion includes a top opening that is circular in shape and has a diameter D, the relationship between the diameter D of the top opening and the depth x is approximately D=2x+y, where the range of x is from 20 microns to 400 microns, and the range of y is from 5 microns to 30 microns.

### RELATED APPLICATION

This application is a 35 U.S.C. 371 national stage application of PCT International Application No. PCT/CN2022/092031 filed on May 10, 2022, which claims the priority of PCT International Application No. PCT/CN2021/127002, filed on Oct. 28, 2021, the entire disclosures of which are incorporated herein by reference.

### TECHNICAL FIELD

The present disclosure relates to the field of biomedical detection, and in particular to a microfluidic substrate, a microfluidic chip comprising the microfluidic substrate, a method for preparing the microfluidic chip, and a method for using the microfluidic chip.

### BACKGROUND

Polymerase Chain Reaction (PCR) is a molecular biology technique used to amplify specific deoxyribonucleic acid (DNA) fragments, which can replicate a small amount of DNA in large quantities and increase its number significantly. Digital polymerase chain reaction (dPCR) technology is a quantitative analysis technology developed on the basis of PCR that can provide digital DNA quantitative information, and greatly improves the sensitivity and accuracy in combination with the microfluidic technology. In the dPCR technology, the nucleic acid sample is sufficiently diluted so that the number of target molecules (i.e., DNA template) in each reaction unit is less than or equal to one. In each reaction unit, the target molecule is amplified through PCR, and after the amplification, the fluorescent signal of each reaction unit is statistically analyzed, so as to realize the absolute quantitative detection of single-molecule DNA. Because dPCR has the advantages of high sensitivity, strong specificity, high detection throughput, accurate quantification, etc., it is widely used in various fields such as clinical diagnosis, gene instability analysis, single-cell gene expression, environmental microbial detection and prenatal diagnosis.

### SUMMARY

According to an aspect of the present disclosure, a microfluidic substrate is provided. The microfluidic substrate comprises a substrate, the substrate comprising a plurality of microcavity regions arranged in an array, each of the plurality of microcavity regions comprises a first portion and a second portion that are stacked, a depth of the first portion is x, the first portion comprises a top opening with a circular shape and a diameter D, and a relationship between the diameter D of the top opening and the depth x is approximately D=2x+y, where a range of x is from 20 microns to 400 microns, and a range of y is from 5 microns to 30 microns.

In some embodiments, the first portion is a blind hole and the first portion does not penetrate the second portion, and the first portion constitutes a microcavity of the microfluidic substrate.

In some embodiments, the substrate further comprises a third portion which is between any adjacent two microcavity regions in the plurality of microcavity regions and not etched. In each microcavity region, a portion of the substrate which is etched constitutes the first portion, a portion of the substrate which is not etched constitutes the second portion, an orthographic projection of the first portion on the microfluidic substrate overlaps an orthographic projection of the second portion on the microfluidic substrate. The second portion and the third portion are integral.

In some embodiments, the range of x is from 20 microns to 100 microns.

In some embodiments, a shape of the first portion is a curved surface body, and the first portion comprises the top opening, a bottom, and a sidewall connecting the top opening and the bottom, the bottom of the first portion is circular in shape and has a diameter of about x microns.

In some embodiments, a tangent plane at each of at least some points on the sidewall is at a non-perpendicular angle to a reference plane where the microfluidic substrate is located.

In some embodiments, the second portion comprises a bottom opening on a side away from the first portion, the first portion penetrates the second portion to form a through hole, and the through hole constitutes a microcavity of the microfluidic substrate.

In some embodiments, a depth of the second portion is x and a shape of the bottom opening of the second portion is circular, a relationship between a diameter D of the bottom opening and the depth x is approximately D=2x+y.

In some embodiments, the first portion and the second portion have a same shape and are axisymmetric about a symmetry axis, the symmetry axis is parallel to a reference plane where the microfluidic substrate is located.

In some embodiments, y is equal to 10 microns.

In some embodiments, the substrate comprises a first base substrate, the first base substrate comprises a plurality of first portions and a plurality of second portions.

In some embodiments, the substrate comprises a first base substrate and a defining layer on the first base substrate, the defining layer comprises a plurality of first portions and a plurality of second portions.

In some embodiments, the microfluidic substrate further comprises a shielding layer. The shielding layer comprises a plurality of first openings, the plurality of first openings correspond to the plurality of microcavity regions one by one, and an orthographic projection of each of the plurality of microcavity regions on the microfluidic substrate at least partially overlaps an orthographic projection of a first opening corresponding to the microcavity region on the microfluidic substrate. An orthographic projection of the shielding layer on the microfluidic substrate at least partially overlaps an orthographic projection of the defining layer on the microfluidic substrate.

In some embodiments, the microfluidic substrate further comprises a spacing region between any two adjacent microcavity regions in the plurality of microcavity regions and a hydrophobic layer disposed within the spacing region. The hydrophobic layer comprises a plurality of second openings, the plurality of microcavity regions correspond to the plurality of second openings one by one, and an orthographic projection of each microcavity region on the microfluidic substrate falls within an orthographic projection of a second opening corresponding to the microcavity region on the microfluidic substrate.

In some embodiments, a shape of the second opening is circular, and a diameter of the second opening is 5 to 20 microns larger than the diameter of the top opening.

In some embodiments, the microfluidic substrate further comprises a hydrophilic layer. The hydrophilic layer is arranged at least in the plurality of microcavity regions, and an orthographic projection of a portion of the hydrophilic layer arranged in each microcavity region on the microfluidic substrate falls within the orthographic projection of the second opening corresponding to the microcavity region on the microfluidic substrate.

According to another aspect of the present disclosure, a microfluidic chip is provided. The microfluidic chip comprises: a first substrate; a second substrate opposite to the first substrate; the microfluidic substrate described in any of the preceding embodiments, the microfluidic substrate being between the first substrate and the second substrate; and a sealing frame between the first substrate and the second substrate, and an orthographic projection of the microfluidic substrate on the first substrate falls within an orthographic projection of the sealing frame on the first substrate.

In some embodiments, the sealing frame comprises a first side and a second side arranged along a first direction and opposite to each other, and a third side and a fourth side arranged along a second direction different from the first direction and opposite to each other, a shape of the first side and the second side is an arc.

In some embodiments, the microfluidic substrate comprises a first edge and a second edge arranged along the second direction and opposite to each other, a distance between an orthographic projection of the third side of the sealing frame on the first substrate and an orthographic projection of the first edge of the microfluidic substrate on the first substrate is 2 mm to 6 mm, and a distance between an orthographic projection of the fourth side of the sealing frame on the first substrate and an orthographic projection of the second edge of the microfluidic substrate on the first substrate is 2 mm to 6 mm.

In some embodiments, a distance between the microfluidic substrate and the second substrate is 0.1 mm to 0.3 mm.

In some embodiments, the second substrate comprises an inlet hole and an outlet hole, and orthographic projections of the inlet hole and the outlet hole on the first substrate fall within the orthographic projection of the sealing frame on the first substrate.

In some embodiments, the first substrate comprises a second base substrate.

In some embodiments, the first substrate comprises: a second base substrate; and a heating electrode between the second base substrate and the microfluidic substrate. An orthographic projection of the plurality of microcavity regions of the microfluidic substrate on the second base substrate falls within an orthographic projection of the heating electrode on the second base substrate.

In some embodiments, an orthographic projection of the sealing frame on the second base substrate falls within the orthographic projection of the heating electrode on the second base substrate.

In some embodiments, the first substrate further comprises: a first dielectric layer between the second base substrate and the heating electrode; and a second dielectric layer between the heating electrode and the microfluidic substrate.

In some embodiments, the first substrate further comprises a conductive layer between the second base substrate and the first dielectric layer, the conductive layer is electrically connected to the heating electrode through a via in the first dielectric layer.

According to yet another aspect of the present disclosure, a method for preparing a microfluidic chip is provided, the method comprising: providing a first substrate; preparing the microfluidic substrate described in any of the preceding embodiments; fixing a sealing frame and the microfluidic substrate on the first substrate, such that an orthographic projection of the microfluidic substrate on the first substrate falls within an orthographic projection of the sealing frame on the first substrate; placing a second substrate on a side of the sealing frame and the microfluidic substrate away from the first substrate; and performing an encapsulation.

In some embodiments, the preparing the microfluidic substrate described in any of the preceding embodiments, comprises: providing a first base substrate and patterning the first base substrate to form the plurality of microcavity regions.

In some embodiments, the preparing the microfluidic substrate described in any of the preceding embodiments, comprises: providing the first base substrate with a thickness of H; forming a hydrophobic layer on the first base substrate; forming a mask pattern comprising a plurality of exposure holes on a side of the hydrophobic layer away from the first base substrate, each of the plurality of exposure holes having a circular shape and a diameter of y, and a range of y being from 5 microns to 30 microns; etching a portion of the first base substrate exposed by the plurality of exposure holes to a depth of x to form the plurality of microcavity regions, a portion of the first base substrate which is in each microcavity region and is etched constitutes the first portion, a portion of the first base substrate which is in each microcavity region and is not etched constitutes the second portion, the range of x is from 20 microns to 100 microns and x is less than H; and removing the mask pattern.

In some embodiments, the preparing the microfluidic substrate described in any of the preceding embodiments, comprises: providing a first base substrate; applying a defining film on the first base substrate and patterning the defining film to form the plurality of microcavity regions.

According to yet another aspect of the present disclosure, a method for using the microfluidic chip described in any of the preceding embodiments is provided, the method comprising: adding a sample solution into a plurality of microcavities of the microfluidic chip; heating the microfluidic chip to react the sample solution in the plurality of microcavities; and detecting an optical signal emitted by the reacted sample solution in the plurality of microcavities with an optical device.

In some embodiments, the first substrate comprises a second base substrate, and the heating the microfluidic chip described in any of the preceding embodiments, comprises: placing the microfluidic chip in a flat thermal cycler.

In some embodiments, the first substrate comprises a second base substrate and a heating electrode between the second base substrate and the microfluidic substrate, and an orthographic projection of the plurality of microcavities of the microfluidic substrate on the second base substrate falls within an orthographic projection of the heating electrode on the second base substrate, and the heating the microfluidic chip, comprises: applying an electrical signal to the microfluidic chip to drive the heating electrode to heat the plurality of microcavities, and detecting a temperature of the plurality of microcavity regions by a temperature sensor to adjust a current flowing through the heating electrode in real time.

### BRIEF DESCRIPTION OF THE DRAWINGS

In order to more clearly describe the technical solutions in the embodiments of the present disclosure, the drawings that need to be used in the embodiments will be briefly introduced. Obviously, the drawings in the following description are only some embodiments of the present disclosure. For those of ordinary skill in the art, other drawings can also be obtained based on these drawings without making creative efforts.

FIG. 1A schematically illustrates a plan view of a microfluidic substrate according to an embodiment of the present disclosure;

FIG. 1B schematically illustrates a cross-sectional view taken along line AA′ of FIG. 1A;

FIG. 2A schematically illustrates a shape of a microcavity;

FIG. 2B schematically illustrates the relationship between the diameter of the opening and the depth of the microcavity;

FIG. 3A schematically illustrates another shape of the microcavity;

FIG. 3B schematically illustrates yet another shape of the microcavity;

FIG. 3C schematically illustrates still another shape of the microcavity;

FIG. 4A schematically illustrates a cross-sectional view of a portion of a microfluidic substrate according to an embodiment of the present disclosure;

FIG. 4B schematically illustrates a cross-sectional view of a portion of a microfluidic substrate according to an embodiment of the present disclosure;

FIG. 5 schematically illustrates a plan view of a microfluidic chip according to an embodiment of the present disclosure;

FIG. 6 schematically illustrates a plan view of the sealing frame of the microfluidic chip in FIG. 5;

FIG. 7 schematically illustrates a plan view of the second substrate of the microfluidic chip in FIG. 5;

FIG. 8A schematically illustrates a cross-sectional view of a portion of the first substrate of the microfluidic chip in FIG. 5;

FIG. 8B schematically illustrates a cross-sectional view of a portion of the first substrate of the microfluidic chip in FIG. 5;

FIG. 9A schematically illustrates a cross-sectional view of a portion of a microfluidic chip according to an embodiment of the present disclosure;

FIG. 9B schematically illustrates a cross-sectional view of a portion of another microfluidic chip according to an embodiment of the present disclosure;

FIG. 9C schematically illustrates a cross-sectional view of a portion of another microfluidic chip according to an embodiment of the present disclosure;

FIG. 9D schematically illustrates a cross-sectional view of a portion of another microfluidic chip according to an embodiment of the present disclosure;

FIG. 9E schematically illustrates a cross-sectional view of a portion of another microfluidic chip according to an embodiment of the present disclosure;

FIG. 9F schematically illustrates a cross-sectional view of a portion of another microfluidic chip according to an embodiment of the present disclosure;

FIG. 10 schematically illustrates a flowchart of a method for preparing a microfluidic chip according to an embodiment of the present disclosure;

FIG. 11 schematically illustrates a mask pattern used in the process of preparing the microfluidic chip;

FIG. 12 schematically illustrates a flowchart of a method for using a microfluidic chip according to an embodiment of the present disclosure; and

FIG. 13 illustrates a fluorescent image of a microfluidic chip irradiated by a light source according to an embodiment of the present disclosure.

### DETAILED DESCRIPTION OF THE DISCLOSURE

The following will clearly and completely describe the technical solutions in the embodiments of the present disclosure with reference to the drawings in the embodiments of the present disclosure. Apparently, the described embodiments are only some, but not all, of the embodiments of the present disclosure. Based on the embodiments in the present disclosure, all other embodiments obtained by those of ordinary skill in the art without making creative efforts belong to the protection scope of the present disclosure.

Digital polymerase chain reaction (dPCR) is a quantitative analysis method that provides digital DNA quantification information, and has illustrated significant advantages in many fields since it was proposed. With the emergence of microfluidic technology and its rapid development in recent years, the combination of microfluidic technology and dPCR technology significantly improves the sensitivity and accuracy of detection. Due to the advantages of high sensitivity, high integration, high automation, and high-throughput detection, the digital microfluidic chip based on dPCR technology has great technical advantages and commercial prospects in research fields such as single cell analysis, early diagnosis of cancer, and prenatal diagnosis.

However, the inventors of the present application have found that there are still problems in the digital microfluidic chip based on the microcavity structure, such as how to ensure that the sample solution can be distributed to each microcavity of the microfluidic chip, how to reduce the air bubbles generated during the sample injection process, how to improve the stability of the microfluidic chip during the heating process, and how to realize the precise control of the fluid, etc.

In view of this, embodiments of the present disclosure provide a microfluidic substrate, a microfluidic chip comprising the microfluidic substrate, a method for preparing the microfluidic chip, and a method for using the microfluidic chip, so as to overcome the above-mentioned problems.

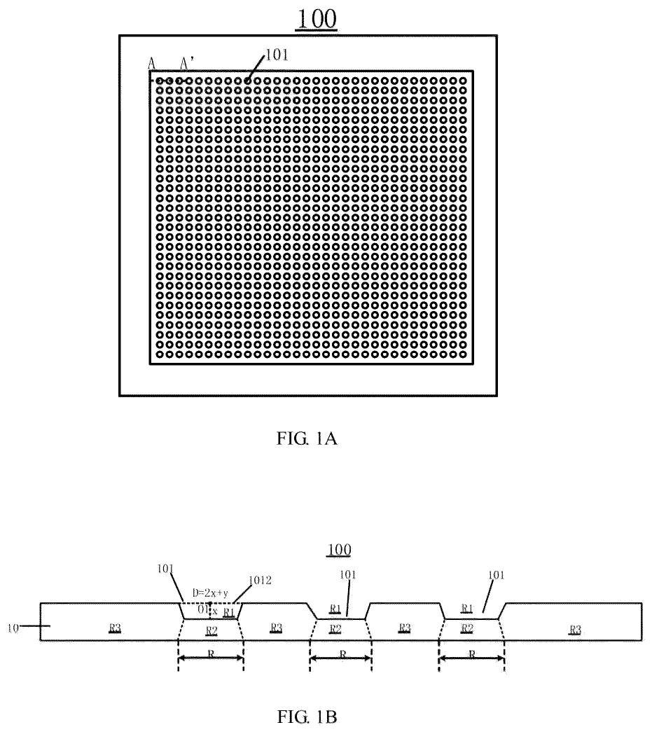

FIG. 1A illustrates a plan view of a microfluidic substrate 100 according to an embodiment of the present disclosure, and FIG. 1B illustrates a partial cross-sectional view taken along line AA′ in FIG. 1A. As illustrated in FIG. 1A and FIG. 1B, the microfluidic substrate 100 comprises a substrate 10, and the substrate 10 comprises a plurality of microcavity regions R arranged in an array (only three microcavity regions R are illustrated in FIG. 1B as an example), each microcavity region R comprises a first portion R1 and a second portion R2 that are stacked. The depth of the first portion R1 is x. The first portion R1 comprises a top opening 1012 with a shape of circular (the center of the circle is O1) and a diameter D. The relationship between the diameter D of the top opening 1012 and the depth x is approximately D=2x+y, where the range of x is from 20 microns to 400 microns, such as 20 microns, 50 microns, 100 microns, 150 microns, 210 microns, 250 microns, 300 microns, 350 microns, 400 microns, etc.; the range of y is from 5 microns to 30 microns, such as 5 microns, 10 microns, 15 microns, 20 microns, 25 microns, 30 microns, etc.

The first portion R1 and the second portion R2 determine the structure and shape of the microcavity in each microcavity region R of the microfluidic substrate 100, the microcavity is the reaction cavity of the microfluidic substrate 100, which is used to accommodate the sample solution, to provide space for reaction (such as PCR reaction) of the sample solution. The top opening 1012 of the first portion R1 refers to the top opening of the microcavity, through which the sample solution enters the microcavity.

In the formula D=2x+y, x is the depth of the first portion R1, and y is the diameter of the exposure hole of the mask pattern used in the preparation process of the microfluidic substrate 100. The smaller the value of y, the higher the precision required for the preparation process, so the greater the difficulty. The exposure hole of the mask pattern is circular in shape and has a diameter of y microns. The microcavity is usually formed by etching a substrate (such as a base substrate or a defining layer above a base substrate). Due to the isotropy of the etching, during the process of etching to form a microcavity, when the etching depth is x microns, the diameter D of the top opening 1012 of the microcavity is about 2x+y microns. This formula roughly expresses the effect of the size design of the exposure hole of the mask pattern on the diameter of the top opening 1012 of the microcavity in isotropic wet etching. In the formula D=2x+y, the coefficient “2” means that the lateral etching speed of the substrate is twice the longitudinal etching speed of the substrate during the etching process. This coefficient is related to factors such as the material of the substrate and the choice of etchant. Therefore, when changing the material of the substrate and/or changing the composition of the etchant, the coefficient will change accordingly. For example, when the material of the substrate and/or the composition of the etchant are changed, the formula D=2x+y can also become D=3x+y, D=4x+y, D=5x+y, D=6x+y etc. The mask pattern will be described in more detail later, and will not be repeated here.

As mentioned above, the first portion R1 and the second portion R2 determine the structure and shape of the microcavity in each microcavity region R of the microfluidic substrate 100. When the first portion R1 is a blind hole and the first portion R1 does not penetrate the second portion R2, the first portion R1 constitutes a microcavity of the microfluidic substrate 100, and in this case, the microcavity is a blind hole. That is to say, in the process of etching the substrate to form the microcavity, the portion which is etched and removed in the microcavity region R is the first portion R1 of the microcavity region R, and the first portion R1 constitutes the blind hole microcavity of the microfluidic substrate 100; the portion which is not etched in the microcavity region R is the second portion R2 of the microcavity region R. When the second portion R2 comprises a bottom opening away from a side of the first portion R1 and the first portion R1 penetrates the second portion R2, the first portion R1 and the second portion R2 are formed as a through hole, the first portion R1 and the second portion R2 constitute a microcavity of the microfluidic substrate 100, in this case, the microcavity is a through hole. That is to say, in the process of etching the substrate to form the microcavity, in the microcavity region R, the entire thickness of the substrate is etched to form a through hole, the first portion R1 in the microcavity region R constitutes the upper portion of the through hole, and the second portion R2 in the microcavity region R constitutes the lower portion of the through hole.

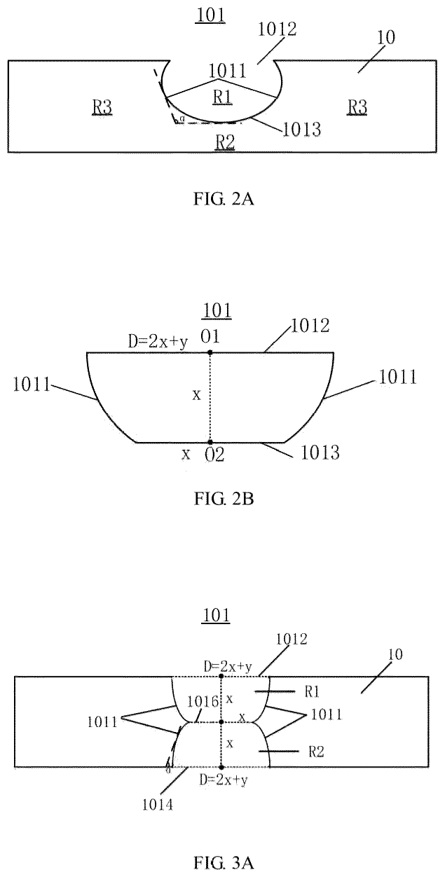

FIG. 2A illustrates a microcavity 101 of a shape as an example, and the microcavity 101 is a blind hole. In this case, the first portion R1 is the microcavity 101. The substrate 10 also comprises a third portion R3 which is not etched and located between any two adjacent microcavity regions R among the plurality of microcavity regions R. In each microcavity region R, the portion of the substrate 10 that is etched and removed constitutes the first portion R1 of the blind hole type, and the portion of the substrate 10 that is not etched and removed constitutes the second portion R2. The orthographic projection of the first portion R1 on the microfluidic substrate 100 overlaps with the orthographic projection of the second portion R2 on the microfluidic substrate 100, and the second portion R2 and the third portion R3 are integral. In other words, when preparing the microcavity illustrated in FIG. 2A, in each microcavity region R, a portion of the substrate 10 is etched to form the first portion R1, which constitutes the blind hole microcavity of the microfluidic substrate 100, while the second portion R2 located directly below the first portion R1 and the third portion R3 located between adjacent microcavities of the substrate 10 are not etched, so that the second portion R2 and the third portion R3 are integral and have the same material. In the case that the microcavity 101 is a blind hole, the microcavity 101 comprises the top opening 1012, a bottom 1013 and a sidewall 1011, and the sidewall 1011 connects the top opening 1012 and the bottom 1013. The sidewall 1011 of the microcavity 101 together with the top opening 1012 and the bottom 1013 constitute the reaction cavity of the microcavity 101 to accommodate the sample solution. It should be noted that, in the present application, the term “sidewall of the microcavity” refers to all walls surrounding the microcavity. Any point on the sidewall 1011 of the microcavity 101 forms an angle α with the reference plane where the microfluidic substrate 100 is located, and a is not equal to 90 degrees. As illustrated in the figure, the microcavity 101 comprises the sidewall 1011, the top opening 1012 and the bottom 1013, and the sidewall 1011 connects the top opening 1012 and the bottom 1013. The blind-hole microcavity 101 can have various suitable shapes, comprising but not limited to a curved surface body, a regular prism and so on. For example, in an example, the blind-hole microcavity 101 may be approximately “bowl-shaped.”

By designing the microcavity 101 as a blind hole, after the sample solution enters the microcavity 101, it can be stably kept in the microcavity and not easily taken out of the microcavity during the detection process. In addition, if air bubbles are generated when the sample solution enters the microcavity 101, the microcavity 101 can absorb these air bubbles on the sidewall 1011 to avoid mixing the air bubbles in the sample solution in the cavity, thereby avoiding affecting the subsequent fluorescence detection of the sample solution.

FIG. 2B illustrates the microcavity 101 in a “bowl shape”. As illustrated in the figure, the microcavity 101 comprises the sidewall 1011, the top opening 1012 and the bottom 1013, and the sidewall 1011 connects the top opening 1012 and the bottom 1013. The shape of the top opening 1012 is circular, the center of the top opening 1012 is O1, and the diameter of the top opening 1012 is D. The depth of the microcavity 101 is x. The relationship between the diameter D of the top opening 1012 of the microcavity 101 and the depth x of the microcavity 101 can roughly be considered as D=2x+y, the range of y is from 5 microns to 30 microns, such as 5 microns, 10 microns, 12 microns, 15 microns, 20 microns, 25 microns, 30 microns, etc. y is the diameter of the exposure hole of the mask pattern used in the preparation process, the smaller the value of y, the higher the precision required for the preparation process, and thus the greater the difficulty. The shape of the exposure hole of the mask pattern is circular and the diameter is y micron. Due to the isotropy in the etching process, during the process of etching the base substrate to form a blind hole microcavity, when the etching depth is x microns, the diameter D of the top opening 1012 of the microcavity 101 is about 2x+y microns. This formula roughly expresses the effect of the size design of the exposure hole of the mask pattern on the diameter of the top opening 1012 of the microcavity 101 in isotropic wet etching. As mentioned above, in the formula D=2x+y, the coefficient “2” means that the lateral etching speed of the substrate is twice the longitudinal etching speed of the substrate during the etching process, and this coefficient is related to factors such as the material of the substrate and the choice of etchant. Therefore, when changing the material of the substrate and/or changing the composition of the etchant, the coefficient will change accordingly. For example, when the material of the substrate and/or the composition of the etchant are changed, the formula D=2x+y can also become D=3x+y, D=4x+y, D=5x+y, D=6x+y etc. The mask pattern will be described in more detail later, and will not be repeated here. It should be noted that the phrase “the relationship between the diameter D of the top opening 1012 of the microcavity 101 and the depth x of the microcavity 101 can roughly be considered as D=2x+y” should be understood as that the value of D is substantially equal to 2x+y, but certain numerical deviations due to preparation process errors should be allowed. In an example, the diameter D of the top opening 1012 of the microcavity 101 is equal to 2x+y, and y is equal to 10 microns.

In some embodiments, the range of x is from 20 microns to 100 microns, such as 20 microns, 30 microns, 40 microns, 50 microns, 60 microns, 70 microns, 80 microns, 90 microns, 100 microns, and so on. In some embodiments, as illustrated in FIG. 2B, the shape of the bottom 1013 of the microcavity 101 is circular, the center of which is 02, and the diameter of the bottom 1013 is approximately equal to x microns. In some embodiments, the diameter of the top opening 1012 of the microcavity 101 is 50-200 microns, such as 50 microns, 80 microns, 110 microns, 125 microns, 150 microns, 180 microns, 200 microns and so on.

As illustrated in FIG. 2B, the sidewall 1011 of the microcavity 101 has a curvature and is relatively smooth, while the bottom 1013 of the microcavity 101 is relatively flat. With such a design, the sample solution can more easily enter the microcavity 101 along the smooth and curved sidewall 1011, and it is not easy to leave air bubbles at the bottom 1013.

The microcavity 101 can be a blind hole or a through hole, and can have various suitable shapes. The tangent plane at each of at least some points on the sidewall 1011 of each microcavity 101 forms a non-perpendicular angle with the reference plane where the microfluidic substrate 100 is located. The definition of “tangent plane” in mathematics textbooks is that, under certain conditions, there are infinitely many curves passing through a point M on a curved surface, and each curve has a tangent line at the point M, under certain conditions, these tangent lines are in a same plane, which is referred as the tangent plane of the curved surface at the point M, and the point M is referred as the tangent point. Therefore, the phrase “the tangent plane at each of at least some points on the sidewall 1011 of each microcavity 101 forms a non-perpendicular angle with the reference plane where the microfluidic substrate 100 is located” means that at least a portion of the sidewall 1011 of each microcavity 101 is not perpendicular to the reference plane (such as a horizontal plane) where the microfluidic substrate 100 is located. For example, all portions on the sidewall 1011 of the microcavity 101 may not be perpendicular to the reference plane, or one or more portions on the sidewall 1011 of the microcavity 101 may not be perpendicular to the reference plane. In other words, at least a portion of the sidewall 1011 of each microcavity 101 has an inclination angle relative to the reference plane, and the inclination angle can be, for example, an acute angle or an obtuse angle. In the related technology, the sidewall of the microcavity is usually perpendicular to the reference plane where the microfluidic substrate is located. Such a steep sidewall is unfavorable for the sample solution to enter the microcavity, causing the sample solution to enter the microcavity very slowly or even stagnate on the surface of the microfluidic substrate, thereby reducing the sampling efficiency and even causing waste of the tiny amount of sample solution. However, in an embodiment of the present disclosure, by making at least a portion of the sidewall 1011 of the microcavity 101 not perpendicular to the reference plane where the microfluidic substrate 100 is located, the slope of the sidewall 1011 of the microcavity 101 relative to the reference plane can be reduced, which is conducive to making the sample solution quickly enter the inside of each microcavity 101 along the sidewall 1011 without stagnation on the surface of the microfluidic substrate 100, thereby further promoting the distribution of the sample solution into each microcavity 101, improving the injection efficiency, and improving the utilization of the sample solution.

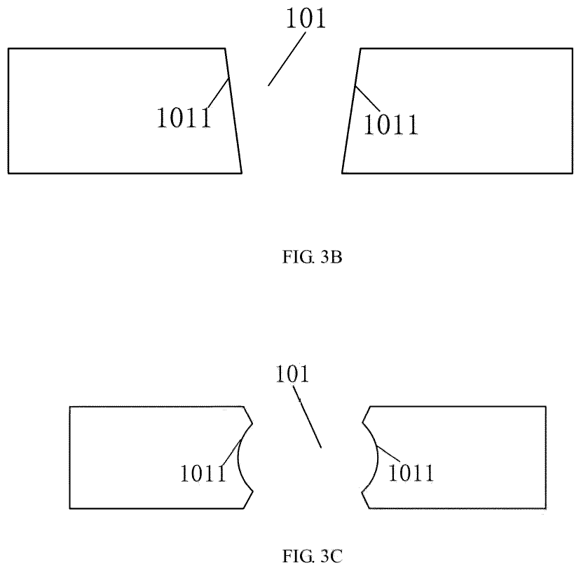

FIG. 3A illustrates a microcavity 101 of a shape as an example, and the microcavity 101 is a through hole. In the case that the microcavity 101 is a through hole, the first portion R1 constitutes the upper portion of the through hole, the second portion R2 constitutes the lower portion of the microcavity, and the first portion R1 penetrates the second portion R2 to form a through hole microcavity. In the case that the microcavity 101 is a through hole, the microcavity 101 comprises a top opening 1012, a bottom opening 1014 and a sidewall 1011, and the sidewall 1011 connects the top opening 1012 and the bottom opening 1014. In this application, the term “a top opening of the microcavity” refers to an opening through which the sample solution enters the microcavity. The term “a bottom opening of the microcavity” refers to an opening opposite to the top opening of the microcavity, and the bottom opening exists only when the microcavity is a through hole. The sidewall 1011 of the microcavity 101 forms an angle α with the reference plane where the microfluidic substrate is located, and α is not equal to 90 degrees.

The microcavity 101 illustrated in FIG. 3A can be regarded as a combination of two “bowl-shaped” microcavities illustrated in FIG. 2B. The shape of the first portion R1 is “bowl-shaped”, and the shape of the second portion R2 is an inverted “bowl-shaped”. The shapes of the first portion R1 and the second portion R2 are axisymmetric about a symmetry axis, which is parallel to the reference plane where the microfluidic substrate 100 is located. As illustrated in the figure, the top opening 1012 of the first portion R1 of the microcavity 101 is circular (the center is O1), the diameter of the top opening 1012 is D, the depth of the first portion R1 is x. The relationship between the diameter D of the top opening 1012 and the depth x is roughly D=2x+y, the range of x is from 20 microns to 400 microns, and the range of y is from 5 microns to 30 microns. The bottom opening 1014 of the second portion R2 of the microcavity 101 is circular (the center is O1), the diameter of the bottom opening 1014 is D, and the depth of the second portion R2 is x. The relationship between the diameter D of the bottom opening 1014 and the depth x is roughly D=2x+y, the range of x is from 20 microns to 400 microns, and the range of y is from 5 microns to 30 microns. As mentioned above, in the formula D=2x+y, the coefficient “2” means that the lateral etching speed of the substrate is twice the longitudinal etching speed of the substrate during the etching process, and this coefficient is related to factors such as the material of the substrate and the choice of etchant. Therefore, when changing the material of the substrate and/or changing the composition of the etchant, the coefficient will change accordingly. For example, when the material of the substrate and/or the composition of the etchant are changed, the formula D=2x+y can also become D=3x+y, D=4x+y, D=5x+y, D=6x+y etc.

As illustrated in FIG. 3A, the first portion R1 penetrates the second portion R2 of the microcavity 101 through a third opening 1016. In some embodiments, the third opening 1016 has a shape of circular and a diameter of about x microns. The sidewall 1011 of the microcavity 101 has a curvature and is relatively smooth. With such a design, the sample solution can enter the microcavity 101 more easily along the smooth and curved sidewall 1011.

FIG. 3B illustrates a microcavity 101 of another shape as an example, and the microcavity 101 is a through hole. The illustrated microcavity 101 may be in the shape of a truncated cone or a regular truncated prism, and the area of the top opening of the microcavity 101 is larger than the area of the bottom opening of the microcavity 101.

FIG. 3C illustrates a microcavity 101 of yet another shape as an example, and the microcavity 101 is a through hole. The microcavity 101 is composed of a top first portion, a middle second portion and a bottom third portion, and is axisymmetric about the symmetry axis. In an example, the shape of the top first portion and the bottom third portion of the microcavity 101 is a truncated cone or a regular truncated prism, and the shape of the middle second portion is a curved surface body. The definition of “curved surface body” in mathematics textbooks is that, the curved geometry can be called a curved surface body as long as a curved surface participates in the curved geometry, or it can be called a curved solid. The surface of a curved surface body can be entirely composed of curved surface, such as a cylinder, a sphere, etc. The surface of a curved surface body can also be a surface composed of a curved surface and a plane.

FIG. 3A to FIG. 3C illustrate several different shapes of the through-hole microcavity 101 as examples, but do not exhaust all possible shapes of the through-hole microcavity 101. For example, the shape of the microcavity 101 can be freely combined from one or more of a curved surface body (such as a bowl), a truncated cone, a regular truncated prism.

In some embodiments, the shape of the top opening of the through-hole microcavity 101 is circular, and the diameter of the top opening is 50-200 microns, such as 50 microns, 80 microns, 110 microns, 125 microns, 150 microns, 180 microns, 200 microns etc. In some embodiments, the depth of the through-hole microcavity 101 is 300-400 microns, such as 300 microns, 350 microns, 400 microns and so on. Since the through-hole microcavity 101 has a deep depth, it can accommodate more doses of sample solution, and allow more doses of sample solution to react at the same time.

By designing the microcavity 101 as a through hole, under the action of capillary, the sample solution can be smoothly entered into the microcavity 101 without stagnation on the surface of the microfluidic substrate 100, resulting in waste of the sample solution. In addition, the sample solution will inevitably produce some air bubbles during the sample injection process. Utilizing the through-hole design of the microcavity 101, the gas can be discharged from the bottom opening of the microcavity 101, so as to avoid air bubbles remaining inside the microcavity 101, thereby not affecting the subsequent fluorescence detection of the sample solution.

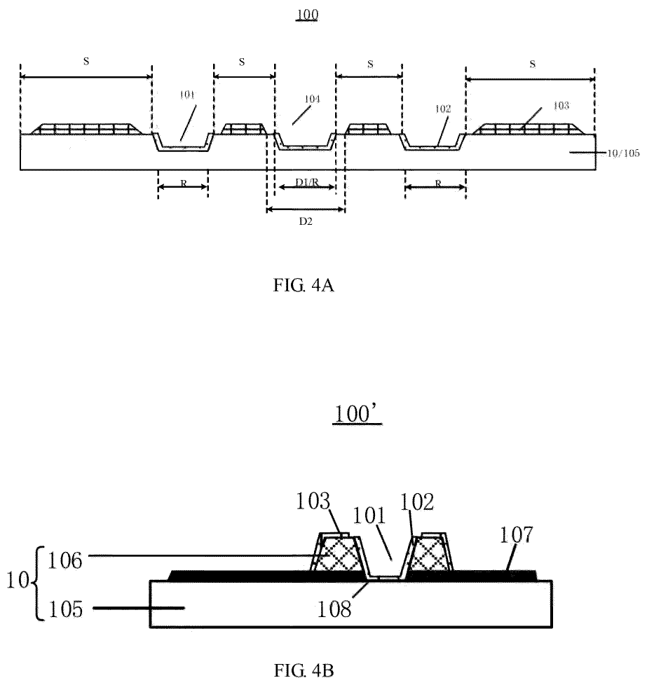

As illustrated in FIG. 4A, the microfluidic substrate 100 may further comprise a spacing region S located between any two adjacent microcavity regions R among the plurality of microcavity regions R, and a hydrophobic layer 103 arranged in the spacing region S. As illustrated in the figure, the hydrophobic layer 103 comprises a plurality of second openings 104, the plurality of microcavities 101 correspond to the plurality of second openings 104 of the hydrophobic layer 103 one by one, and the orthographic projection of each microcavity region R on the microfluidic substrate 100 falls within the orthographic projection of a second opening 104 corresponding to the microcavity region R on the microfluidic substrate 100. The microfluidic substrate 100 may further comprise a hydrophilic layer 102, and the hydrophilic layer 102 is located at least in the plurality of microcavity regions R. For example, when the microcavity 101 is a blind hole, the hydrophilic layer 102 covers at least the sidewall 1011 and the bottom 1013 of the microcavity 101. When the microcavity 101 is a through hole, the hydrophilic layer 102 covers at least the sidewall 1011 of the microcavity 101. The orthographic projection of the part of the hydrophilic layer 102 located in each microcavity region R on the microfluidic substrate 100 falls within the orthographic projection of a second opening 104 corresponding to the microcavity region R on the microfluidic substrate 100. It should be noted that although the hydrophilic layer 102 is only located in each microcavity region R in the figure, this is only an example. In the alternative embodiments, the hydrophilic layer 102 is not only located in each microcavity region R, but also located in some regions in the spacing region S. By arranging the hydrophobic layer 103 in the spacing region S between two adjacent microcavities 101 of the microfluidic substrate 100, the hydrophobic performance of the outer region of the microcavity 101 can be improved. The hydrophilic layer 102 is arranged inside the microcavity 101 (at least on the sidewall 1011), the hydrophilic performance of the inside of the microcavity 101 can be improved. Therefore, the hydrophilic layer 102 and the hydrophobic layer 103 can jointly adjust the surface contact angle of the droplet of the sample solution. In the case that no external driving force is applied to the sample solution, the sample solution can automatically enter each microcavity 101 of the microfluidic substrate 100 based on the capillary phenomenon, thereby improving the uniformity of distribution of the sample solution and avoiding the cross-flow of the sample solution. By arranging the microcavities in the microfluidic substrate 100, the amount of sample solution flowing into each microcavity 101 can be substantially the same by designing uniformly sized microcavities 101, so that precise control of the sample solution can be achieved. The orthographic projection of the portion of the hydrophilic layer 102 located in each microcavity 101 on the microfluidic substrate 100 falls within the orthographic projection of a second opening 104 corresponding to the microcavity 101 on the microfluidic substrate 100. That is to say, near each microcavity 101, the hydrophilic layer 102 and the hydrophobic layer 103 have a certain boundary distance.

The orthographic projection of each microcavity 101 on the microfluidic substrate 100 falls within the orthographic projection of a second opening 104 corresponding to the microcavity 101 on the microfluidic substrate 100. In some embodiments, the shape of the top opening of each microcavity 101 is circular, and the shape of a second opening 104 corresponding to the microcavity 101 is also circular. As illustrated in FIG. 4A, the diameter of the top opening of the microcavity 101 is D1, and the diameter of the second opening 104 of the hydrophobic layer 103 is D2. In some examples, the diameter D2 of the second opening 104 of the hydrophobic layer 103 is 5 to 20 microns larger than the diameter D1 of the top opening of the microcavity 101.

As illustrated in FIG. 4A, in some embodiments, the substrate 10 may comprise a first base substrate 105 comprising the aforementioned plurality of microcavities 101, and each microcavity 101 may be a through hole or a blind hole. In other words, the aforementioned plurality of microcavities 101 are formed by patterning the first base substrate 105. In some embodiments, the thickness of the first base substrate 105 is 0.3-0.7 mm, such as 0.3 mm, 0.4 mm, 0.5 mm, 0.6 mm, 0.7 mm, and so on. The first base substrate 105 may be formed of various suitable materials, comprising but not limited to glass, quartz, silicon, and the like.

FIG. 4B illustrates a cross-sectional view of a portion of the microfluidic substrate 100′. Except for the arrangement of the microcavities 101 and the shielding layer 107, the structure of the microfluidic substrate 100′ in FIG. 4B is basically the same as that of the microfluidic substrate 100 in FIG. 4A. For example, the microfluidic substrate 100′ also comprises a hydrophilic layer 102 and a hydrophobic layer 103, and the arrangement of the hydrophilic layer 102 and the hydrophobic layer 103 in FIG. 4B is the same as that of the hydrophilic layer 102 and the hydrophobic layer 103 in FIG. 4A. Therefore, the structure and effect of the hydrophilic layer 102 and the hydrophobic layer 103 in FIG. 4B can refer to the description of FIG. 4A, and will not be repeated here. For the sake of brevity, the similarities between the microfluidic substrate 100′ in FIG. 4B and the microfluidic substrate 100 in FIG. 4A will not be described repeatedly, and only the differences will be introduced below.

As illustrated in FIG. 4B, the substrate 10 comprises a first base substrate 105 and a defining layer 106, the defining layer 106 is located between the first base substrate 105 and the hydrophobic layer 103, the defining layer 106 comprises the aforementioned multiple microcavities 101, each microcavity 101 may be a through hole or a blind hole. That is to say, different from FIG. 4A, the microcavity 101 is not formed by patterning the first base substrate 105, but is formed by patterning the defining layer 106. The defining layer 106 may be composed of various suitable materials, comprising but not limited to photoresist.

As illustrated in FIG. 4B, the microfluidic substrate 100′ may further comprise a shielding layer 107 comprising a plurality of first openings 108, and the plurality of first openings 108 correspond to the plurality of microcavities 101 one by one. The orthographic projection of each microcavity 101 on the microfluidic substrate 100′ at least partially overlaps the orthographic projection of a first opening 108 corresponding to the microcavity 101 on the microfluidic substrate 100′, and the orthographic projection of the shielding layer 107 on the microfluidic substrate 100′ at least partially overlaps the orthographic projection of the defining layer 106 on the microfluidic substrate 100′. In an example, the orthographic projection of the defining layer 106 on the microfluidic substrate 100′ completely falls within the orthographic projection of the shielding layer 107 on the microfluidic substrate 100′. The shielding layer 107 may be made of any appropriate material, as long as the material can shield light or absorb light, and the embodiment of the present disclosure does not specifically limit the material of the shielding layer 107. In some embodiments, the material of the shielding layer 107 is an opaque material, such as an opaque metal. In some examples, the material of the shielding layer 107 is a black matrix (BM) commonly used in the display field.

When the material of the defining layer 106 is photoresist, the defining layer 106 usually emits undesired fluorescence after being irradiated by the excitation light due to its inherent material properties, and the undesired fluorescence will interfere with the fluorescence signal emitted by the sample solution in the microcavities 101. However, in the embodiment of the present disclosure, by disposing the shielding layer 107 and making the orthographic projection of the shielding layer 107 on the microfluidic substrate 100′ at least partially overlap the orthographic projection of the defining layer 106 on the microfluidic substrate 100′, when the excitation light is irradiated to the microcavity 101 through the first opening 108 of the shielding layer 107, the shielding layer 107 can at least partially shield the defining layer 106 so that the defining layer 106 is not irradiated by the excitation light, thereby preventing the defining layer 106 from being irradiated by the excitation light to generate interference fluorescence. In this way, the excitation light can only excite the sample solution in the microcavity 101 through the first opening 108. Therefore, with such an arrangement, the fluorescence interference caused by the defining layer 106 can be reduced or even avoided, so that the fluorescence signal emitted by the sample solution in the microcavity 101 can be accurately identified by the detector, so that the reaction signal can be read more sensitively and accurately, the fluorescence detection accuracy of the sample solution can be improved, and image data support can be provided for the data analysis of the subsequent nucleic acid amplification reaction. In addition, with such an arrangement, clearer microwell array imaging can be achieved, detection errors caused by false positives can be reduced, and interference between different channels in the multi-channels fluorescence signal detection process can be well avoided.

It should be noted that although FIG. 4B shows that the shielding layer 107 is located between the first base substrate 105 and the defining layer 106, this is only an example, and the shielding layer 107 may also be located at other positions. In some embodiments, the shielding layer 107 may be located on a side of the first base substrate 105 away from the defining layer 106, that is, located on the back side of the first base substrate 105. In an alternative embodiment, the shielding layer 107 may be located on the side of the defining layer 106 facing away from the first base substrate 105 and attached to the side surfaces of the defining layer 106 and the surface of the defining layer 106 facing away from the first base substrate 105. In an alternative embodiment, the shielding layer 107 is not only positioned between the first base substrate 105 and the defining layer 106, i.e., attached to the surface of the defining layer 106 close to the first base substrate 105, but also located on a side of the defining layer 106 away from the first base substrate 105, i.e., attached to the side surfaces of the defining layer 106 and the surface of the defining layer 106 away from the first base substrate 105. That is, the shielding layer 107 surrounds the defining layer 106 from all sides.

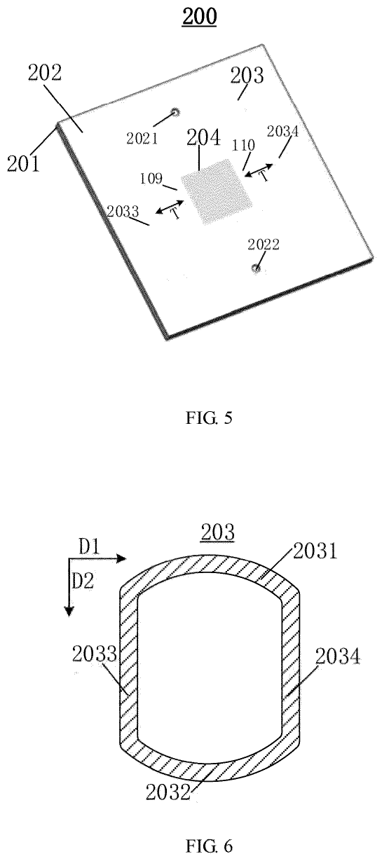

According to another aspect of the present disclosure, a microfluidic chip is provided. FIG. 5 illustrates a schematic plan view of the microfluidic chip 200. As illustrated in FIG. 5, the microfluidic chip 200 comprises: a first substrate 201; a second substrate 202 facing to the first substrate 201; a microfluidic substrate 204, which is between the first substrate 201 and the second substrate 202 and may be the microfluidic substrate 100 or 100′ described in any of the previous embodiments; and a sealing frame 203 between the first substrate 201 and the second substrate 202, the orthographic projection of the microfluidic substrate 204 on the first substrate 201 falling within the orthographic projection of the sealing frame 203 on the first substrate 201.

FIG. 6 illustrates a schematic plan view of the sealing frame 203. The sealing frame 203 is configured to keep an appropriate distance between the first substrate 201 and the second substrate 202 and keep the microfluidic chip 200 in a sealed state. In some embodiments, the sealing frame 203 is an elastic sealing frame. In some embodiments, the sealing frame 203 is made of silicone material, has a certain shape by die-cutting, and surrounds the periphery of the microfluidic substrate 204. As illustrated in FIG. 6, the sealing frame 203 comprises a first side 2031 and a second side 2032 arranged along a first direction D1 and opposite to each other, and a third side 2033 and a fourth side 2034 arranged along a second direction D2 different from the first direction D1 and opposite to each other. The shape of the first side 2031 and the second side 2032 is an arc. In an example, the shape of the first side 2031 and the second side 2032 of the sealing frame 203 is a circular arc. This arc or circular arc design is beneficial to promote the flow and gathering together of the sample solution in the microfluidic chip 200, and can avoid residual air bubbles in the microfluidic chip 200 during the sample injection process.

Referring to FIGS. 5 and 6, the microfluidic substrate 204 comprises a first edge 109 and a second edge 110 arranged along the second direction D2 and opposite to each other. The third side 2033 of the sealing frame 203 is at a distance from the first edge 109 of the microfluidic substrate 204, and the fourth side 2034 of the sealing frame 203 is at a distance from the second edge 110 of the microfluidic substrate 204. In some embodiments, the distance between the orthographic projection of the third side 2033 of the sealing frame 203 on the first substrate 201 and the orthographic projection of the first edge 109 of the microfluidic substrate 204 on the first substrate 201 is 2 mm to 6 mm, such as 2 mm, 3 mm, 4 mm, 5 mm, 6 mm; the distance between the orthographic projection of the fourth side 2034 of the sealing frame 203 on the first substrate 201 and the orthographic projection of the second edge 110 of the microfluidic substrate 204 on the first substrate 201 is 2 mm to 6 mm, such as 2 mm, 3 mm, 4 mm, 5 mm, 6 mm. In some embodiments, the area of the region where the multiple microcavities 101 of the microfluidic substrate 204 are located is 15×15 mm², and the area of the microfluidic substrate 204 is 17×17 mm². That is, there is a distance between the four edges of the microfluidic substrate 204 and the region where the microcavities 101 are located. In this way, the microcavity region can be avoided from cutting during the cutting process of the microfluidic chip 200, and sufficient space can be reserved for subsequent packaging.

In some embodiments, the thickness of the sealing frame 203 is 0.4-0.8 mm, such as 0.4 mm, 0.5 mm, 0.6 mm, 0.7 mm, 0.8 mm, and so on. The thickness of the sealing frame 203 is greater than the thickness of the microfluidic substrate 204. In some embodiments, the thickness of the sealing frame 203 is greater than that of the microfluidic substrate 204 by 0.1-0.3 mm, for example, greater than 0.1 mm, 0.2 mm, or 0.3 mm. In other words, the distance between the microfluidic substrate 204 and the second substrate 202 is 0.1-0.3 mm By making the distance between the microfluidic substrate 204 and the second substrate 202 small, even if air bubbles are generated during the heating of the microfluidic chip 200, under the surface tension and the extrusion between the microfluidic substrate 204 and the second substrate 202, the air bubbles are also very easy to automatically move to the outer open region so as to be discharged from the microfluidic chip 200, to prevent air bubbles from circulating and flowing with the liquid inside the microfluidic chip 200, affecting the reaction and the subsequent fluorescence detection of the sample.

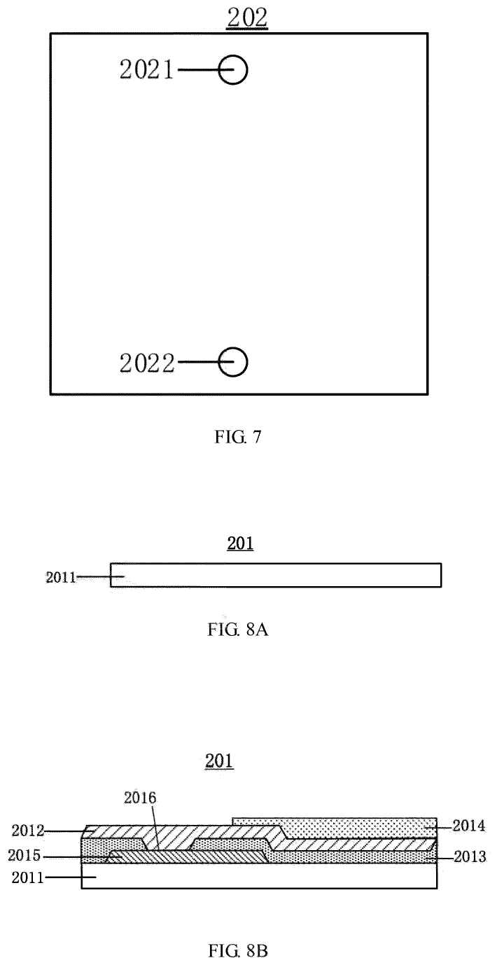

FIG. 7 illustrates a schematic plan view of the second substrate 202. As illustrated in FIG. 7, the second substrate 202 comprises an inlet hole 2021 and an outlet hole 2022, the sample solution is injected into the microcavity 101 of the microfluidic chip 200 through the inlet hole 2021, and the sample solution processed by the microfluidic chip 200 can be transferred to other external device through the outlet hole 2022. The shape of the inlet hole 2021 and the outlet hole 2022 can be circular, and the diameter of the hole is about 0.5-1.5 mm. Referring to FIG. 5 and FIG. 7, the orthographic projections of the inlet hole 2021 and the outlet hole 2022 of the second substrate 202 on the first substrate 201 fall within the orthographic projection of the sealing frame 203 on the first substrate 201.

In some embodiments, the second substrate 202 may be cut from large-sized white glass, and the size of the second substrate 202 may be 40×42 mm². In some embodiments, the thickness of the second substrate 202 is 0.3-0.7 mm, such as 0.3 mm, 0.4 mm, 0.5 mm, 0.6 mm, 0.7 mm, and so on. In some embodiments, the distance between the two side edges of the second substrate 202 along the first direction D1 and the microcavity region is 16 mm, and the distance between the two side edges of the second substrate 202 along the second direction D2 and the microcavity region is 15 mm With such a design, sufficient space can be reserved for the arrangement of the heating electrode of the first substrate 201.

FIG. 8A illustrates a cross-sectional view of a portion of the first substrate 201 in an embodiment. As illustrated in the figure, the first substrate 201 comprises a second base substrate 2011. The second base substrate 2011 may be made of various suitable materials, such as glass. When the microfluidic chip 200 comprises such a first substrate 201, after the packaging of the microfluidic chip 200 is completed, the microcavities 101 of the microfluidic chip 200 can be heated by a flat plate thermal cycler, to make the sample solution in the microcavities 101 react, such as PCR reaction.

FIG. 8B illustrates a cross-sectional view of a portion of the first substrate 201 in another embodiment. As illustrated in the figure, the first substrate 201 comprises: a second base substrate 2011; and a heating electrode 2012 between the second base substrate 2011 and the microfluidic substrate 204. The orthographic projection of the plurality of microcavities 101 of the microfluidic substrate 204 on the second base substrate 2011 falls within the orthographic projection of the heating electrode 2012 on the second base substrate 2011. The heating electrode 2012 is configured to heat the plurality of microcavities 101. The heating electrode 2012 can receive an electrical signal (such as a voltage signal), when a current flows through the heating electrode 2012, heat will be generated, and the heat will be transferred to the microcavity 101 for polymerase chain reaction. For example, the heating electrode 2012 may be made of a conductive material with a relatively high resistivity, so that the heating electrode 2012 can generate a large amount of heat when it is provided with a small electrical signal, so as to improve the energy conversion rate. For example, the heating electrode 2012 may be made of transparent conductive materials, such as indium tin oxide (ITO), tin oxide, etc., or may be made of other suitable materials, such as metal, which is not limited in the embodiments of the present disclosure. By disposing the heating electrode 2012 in the first substrate 201 (for example, integrating the heating electrode 2012 on the second base substrate 2011), the heating of the microcavities 101 can be realized without external heating device and the temperature of the microcavities 101 can be controlled in real time. Therefore, the microfluidic chip 200 comprising the first substrate 201 has higher integration and more precise temperature control, and improves the stability of the microfluidic chip 200 during the heating process.

As illustrated in FIG. 8B, the first substrate 201 may further comprise: a first dielectric layer 2013 between the second base substrate 2011 and the heating electrode 2012 and a second dielectric layer 2014 between the heating electrode 2012 and the microfluidic substrate 204. The first dielectric layer 2013 and the second dielectric layer 2014 may be made of various appropriate materials, and the embodiments of the present disclosure do not limit the specific materials of the first dielectric layer 2013 and the second dielectric layer 2014. In an example, the first dielectric layer 2013 and the second dielectric layer 2014 may be made of SiO₂. The first substrate 201 may further comprise a conductive layer 2015 between the second base substrate 2011 and the first dielectric layer 2013, and the conductive layer 2015 is electrically connected to the heating electrode 2012 through the via 2016 in the first dielectric layer 2013. The conductive layer 2015 is configured to apply an electrical signal (e.g., a voltage signal) to the heating electrode 2012.

The microfluidic chip 200 may have basically the same technical effect as the microfluidic substrate described in the previous embodiments, therefore, for the sake of brevity, the technical effect of the microfluidic chip 200 will not be described here again.

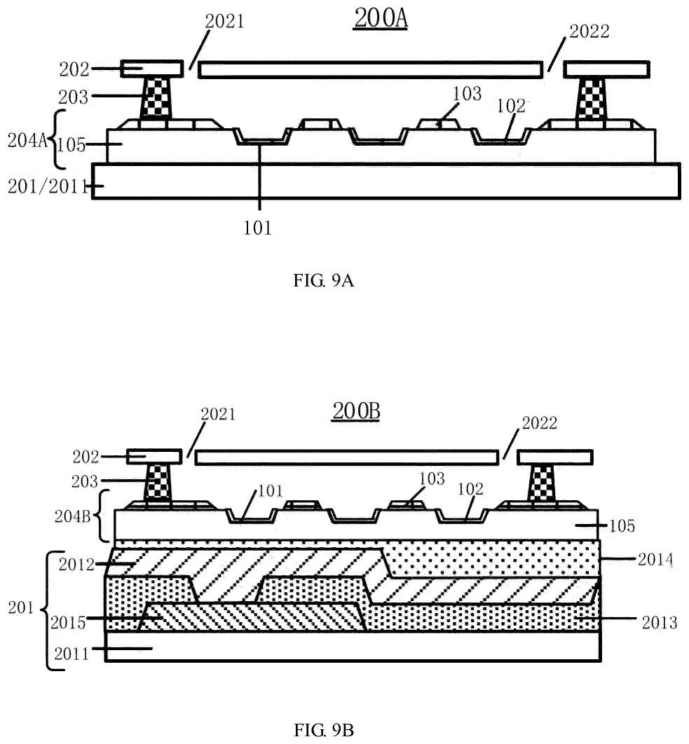

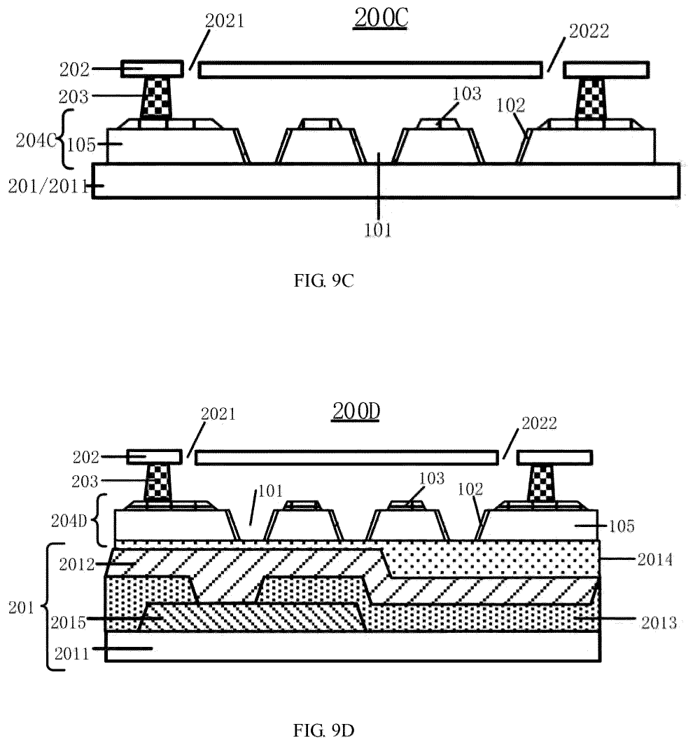

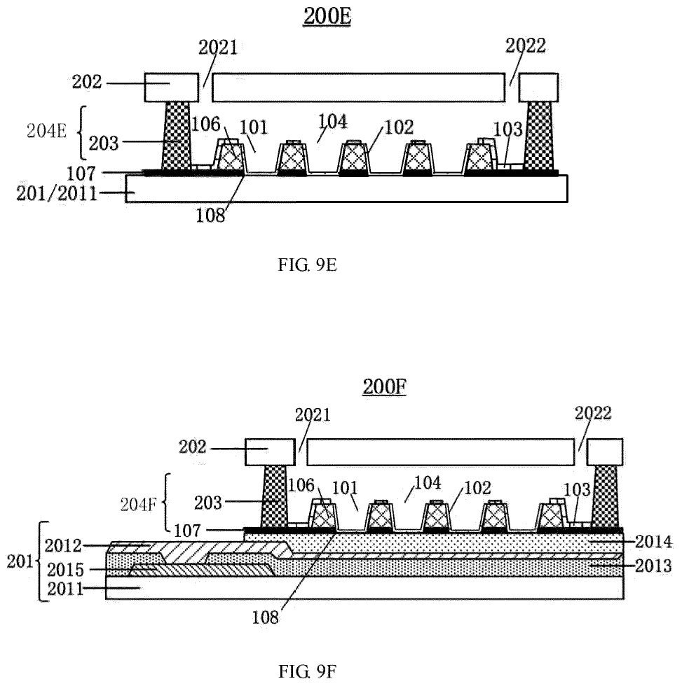

FIGS. 9A-9F illustrate several different microfluidic chips as examples. These microfluidic chips have basically the same structure, for example, they all comprise the first substrate 201, the second substrate 202, the sealing frame 203, and the microfluidic substrate, and the structures of the second substrate 202 and the sealing frame 203 are the same as those of the second substrate 202 and the sealing frame 203 described with respect to FIGS. 5-7. These microfluidic chips differ in the composition of the first substrate 201 and the arrangement of the microcavities 101 in the microfluidic substrate. For the sake of brevity, only the differences between the microfluidic chips are described below.

FIG. 9A illustrates a microfluidic chip 200A, which comprises a first substrate 201, a second substrate 202, a sealing frame 203, and a microfluidic substrate 204A between the first substrate 201 and the second substrate 202. The microfluidic substrate 204A comprises a first base substrate 105, and each microcavity 101 is a blind hole and is formed by patterning the first base substrate 105. For the specific structure of the microfluidic substrate 204A, reference may be made to the foregoing descriptions with respect to FIG. 1B, FIG. 2A, and FIG. 2B. The first substrate 201 is the second base substrate 2011. The microfluidic chip 200A illustrated in FIG. 9A can be abbreviated as “a blind-hole microcavity formed in the first base substrate+a first substrate in the form of the base substrate”.

FIG. 9B illustrates a microfluidic chip 200B, which comprises a first substrate 201, a second substrate 202, a sealing frame 203, and a microfluidic substrate 204B between the first substrate 201 and the second substrate 202. The microfluidic substrate 204B comprises a first base substrate 105, and each microcavity 101 is a blind hole and is formed by patterning the first base substrate 105. For the specific structure of the microfluidic substrate 204B, reference may be made to the foregoing descriptions with respect to FIG. 1B, FIG. 2A, and FIG. 2B. The first substrate 201 comprises a second base substrate 2011, a heating electrode 2012, a first dielectric layer 2013, a second dielectric layer 2014 and a conductive layer 2015. For the specific structure of the first substrate 201, reference may be made to the description of FIG. 8B. As illustrated in the figure, the orthographic projection of the sealing frame 203 on the second base substrate 2011 falls within the orthographic projection of the heating electrode 2012 on the second base substrate 2011. The microfluidic chip 200B illustrated in FIG. 9B may be abbreviated as “a blind-hole microcavity formed in the first base substrate+a first substrate integrated with the heating electrode”.

FIG. 9C illustrates a microfluidic chip 200C, which comprises a first substrate 201, a second substrate 202, a sealing frame 203, and a microfluidic substrate 204C between the first substrate 201 and the second substrate 202. The microfluidic substrate 204C comprises a first base substrate 105, and each microcavity 101 is a through hole and is formed by patterning the first base substrate 105. For the specific structure of the microfluidic substrate 204C, reference may be made to the foregoing descriptions of FIG. 1B and FIGS. 3A-3C. The first substrate 201 is the second base substrate 2011. The microfluidic chip 200C illustrated in FIG. 9C may be abbreviated as “a through-hole microcavity formed in the first base substrate+a first substrate in the form of the base substrate”.

FIG. 9D illustrates a microfluidic chip 200D, which comprises a first substrate 201, a second substrate 202, a sealing frame 203, and a microfluidic substrate 204D between the first substrate 201 and the second substrate 202. The microfluidic substrate 204D comprises a first base substrate 105, and each microcavity 101 is a through hole and is formed by patterning the first base substrate 105. For the specific structure of the microfluidic substrate 204D, reference may be made to the foregoing descriptions of FIG. 1B and FIGS. 3A-3C. The first substrate 201 comprises a second base substrate 2011, a heating electrode 2012, a first dielectric layer 2013, a second dielectric layer 2014 and a conductive layer 2015. For the specific structure of the first substrate 201, reference may be made to the description of FIG. 8B. As illustrated in the figure, the orthographic projection of the sealing frame 203 on the second base substrate 2011 falls within the orthographic projection of the heating electrode 2012 on the second base substrate 2011. The microfluidic chip 200D illustrated in FIG. 9D may be abbreviated as “a through-hole microcavity formed in the first base substrate+a first substrate integrated with the heating electrode”.

FIG. 9E illustrates a microfluidic chip 200E, which comprises a first substrate 201, a second substrate 202, a sealing frame 203, and a microfluidic substrate 204E between the first substrate 201 and the second substrate 202. The microfluidic substrate 204E comprises a defining layer 106 and a shielding layer 107, and each microcavity 101 is a through hole or a blind hole and is formed by patterning the defining layer 106. For the specific structure of the microfluidic substrate 204E, reference may be made to the foregoing description of FIG. 4B. The first substrate 201 is the second base substrate 2011. The microfluidic chip 200E illustrated in FIG. 9E may be abbreviated as “a through-hole or blind-hole microcavity formed in the defining layer+a first substrate in the form of the base substrate”.

FIG. 9F illustrates a microfluidic chip 200F, which comprises a first substrate 201, a second substrate 202, a sealing frame 203, and a microfluidic substrate 204F between the first substrate 201 and the second substrate 202. The microfluidic substrate 204F comprises a defining layer 106 and a shielding layer 107, and each microcavity 101 is a through hole or a blind hole and is formed by patterning the defining layer 106. For the specific structure of the microfluidic substrate 204F, reference may be made to the foregoing description of FIG. 4B. The first substrate 201 comprises a second base substrate 2011, a heating electrode 2012, a first dielectric layer 2013, a second dielectric layer 2014 and a conductive layer 2015. For the specific structure of the first substrate 201, reference may be made to the description of FIG. 8B. As illustrated in the figure, the orthographic projection of the sealing frame 203 on the second base substrate 2011 falls within the orthographic projection of the heating electrode 2012 on the second base substrate 2011. The microfluidic chip 200F illustrated in FIG. 9F may be abbreviated as “a through-hole or blind-hole microcavity formed in the defining layer+a first substrate integrated with the heating electrode”.

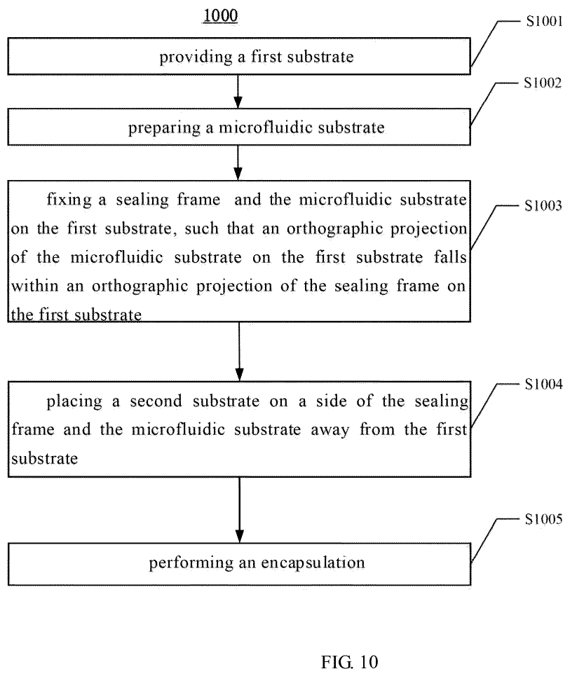

FIG. 10 illustrates a flowchart of a method 1000 for preparing a microfluidic chip, and the method 1000 is suitable for preparing the microfluidic chip described in any of the preceding embodiments. The steps of the method 1000 are as follows.

Step S1001: providing a first substrate 201.

Step S1002: preparing a microfluidic substrate, which may be the microfluidic substrate described in any of the previous embodiments.

Step S1003: fixing a sealing frame 203 and the microfluidic substrate prepared above on the first substrate 201, such that the orthographic projection of the microfluidic substrate on the first substrate 201 falls within the orthographic projection of the sealing frame 203 on the first substrate 201.

Step S1004: placing a second substrate 202 on a side of the sealing frame 203 and the microfluidic substrate away from the first substrate 201.

Step S1005: performing an encapsulation.

The method for preparing the microfluidic chip 200A will be described in detail below by taking the microfluidic chip 200A illustrated in FIG. 9A as an example.

The steps of the preparation of the microfluidic substrate 204A are as follows.

Step 1101: providing a first base substrate 105 and cleaning it. The first base substrate 105 may be made of any suitable material, and in an example, the first base substrate 105 is made of glass. The first base substrate 105 may have any suitable thickness H, and in an example, the thickness H of the first base substrate 105 is 300-700 μm.

Step 1102: preparing a mark on the first base substrate 105 to provide a positioning function for subsequent cutting of the substrate. In an example, the process of forming the mark is as follows: sputtering a metal Mo layer with a thickness of about 2200 Å on the surface of the first base substrate 105. The Mo layer is exposed, developed, and etched using a photolithography process to form the metal mark.

Step 1103: depositing an insulating layer on the surface of the first base substrate 105 on which the metal mark is formed, and performing exposure, development, and etching on the insulating layer to form a hydrophobic layer 103. In an example, the process of forming the hydrophobic layer 103 is as follows: depositing a SiNₓ layer with a thickness of about 3000 Å on the surface of the first base substrate 105. The SiNₓ layer is exposed, developed and etched to form the hydrophobic layer 103. The hydrophobic layer 103 comprises a plurality of second openings 104.

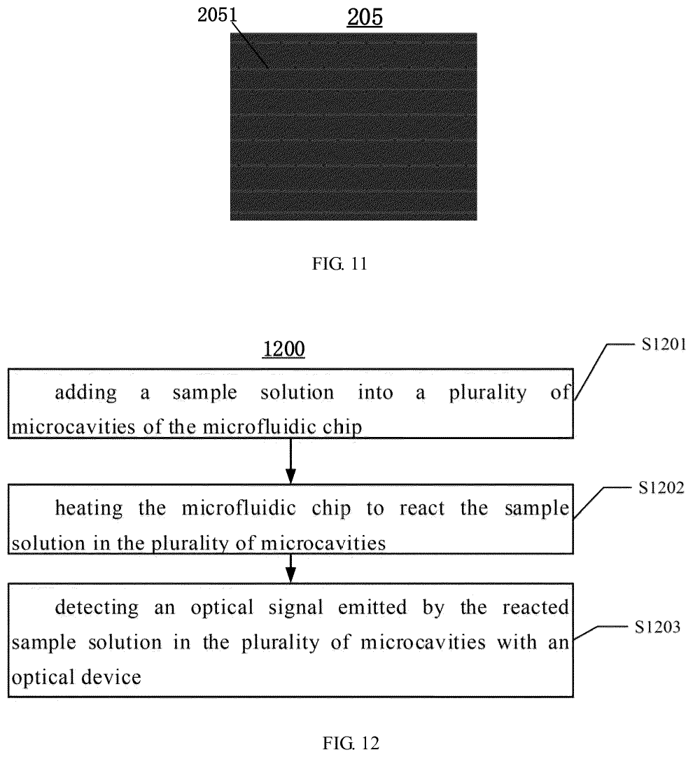

Step 1104: forming a mask pattern on a side of the hydrophobic layer 103 away from the first base substrate 105. The mask pattern is used to define the shape of the microcavity formed by etching and to provide isolation and protection for other parts except the microcavity during the process of etching the microcavity. In an example, the process of forming the mask pattern is as follows: sputtering a metal Mo layer with a thickness of about 2200 Å on the side of the hydrophobic layer 103 away from the first base substrate 105. The Mo layer is exposed, developed, and etched using a photolithography process to form a metal mask pattern 205. The metal mask pattern 205 is illustrated in FIG. 11. The metal mask pattern 205 comprises a plurality of exposure holes 2051 corresponding to the positions of a plurality of microcavities to be formed later, so as to expose regions that need to be etched to form microcavities later. In some embodiments, each exposure hole of the metal mask pattern 205 has a circular shape and has a diameter of y, where the range of y is from 5 μm to 30 μm. In an example, the value of y is 10 μm.

Step 1105: etching the microcavity 101 by wet etching method. The specific steps may be described as follows: immersing the microfluidic substrate 204A formed with the metal mask pattern 205 in an etchant. The concentration of hydrogen fluoride (HF) in the etchant is about 40%, the etching speed is about 3.5 μm/minute, and the surface of the first base substrate 105 facing the metal mask pattern 205 is etched. During the etching, the blades are used to continuously stir the etchant, so that the etchant can etch the first base substrate 105 more uniformly. The etching time needs about 60 minutes to form the microcavity 101, which is a blind hole. The microcavity 101 is formed by wet etching, the shape of the exposure hole 2051 of the metal mask pattern 205 is circular. Due to the isotropic property of wet etching, the shape of the top opening of the microcavity 101 is also generally circular. The diameter of the top opening is about 50-200 μm, such as 50 μm, 80 μm, 100 μm, 120 μm, 150 μm, 200 μm, etc. The depth of the microcavity 101 is about 20-100 μm, such as 20 μm, 40 μm, 60 μm, 80 μm, 100 μm, and so on. For the specific shape of the microcavity 101, reference may be made to the previous descriptions of FIG. 3A and FIG. 3B, and for the sake of brevity, details are not repeated here. The blind hole structure of the microcavity 101 is beneficial to keep the sample solution within the cavity stably during the detection, and makes the sample solution not easily be taken out of the microcavity 101. In an example, when the metal mask pattern 205 is used to etch the first base substrate 105, the relationship between the diameter D of the top opening of the blind-hole microcavity 101 and the etching depth x of the microcavity 101 can be approximately D=2x+y, the range of x is 20-100 μm, and the range of y is 10-30 μm, for example, 10 μm. The microcavity 101 has a smooth sidewall and a flat bottom, which facilitates the sample solution to flow into the microcavity 101 along the smooth sidewall and is not easily taken out, and the generation of air bubbles can be reduced. The orthographic projection of each microcavity 101 on the first base substrate 105 falls within the orthographic projection of a second opening 104 corresponding to the microcavity 101 on the first base substrate 105. In some embodiments, the shape of the second opening 104 of the hydrophobic layer 103 is circular, the shape of the top opening of the microcavity 101 is circular, and the diameter of the second opening 104 of the hydrophobic layer 103 is 5-20 jam larger than the diameter of the top opening of the microcavity 101.

Step 1106: removing the metal mask pattern 205 after the microcavity 101 is etched.

Step 1107: depositing an insulating layer on the surface of the first base substrate 105, performing exposure, development, and etching on the insulating layer to form a hydrophilic layer 102. The hydrophilic layer 102 is only located at the sidewall and bottom of each microcavity 101. In an example, the process of forming the hydrophilic layer 102 is as follows: depositing a SiO₂ layer on the surface of the first base substrate 105, the SiO₂ layer is exposed, developed, and etched to form the hydrophilic layer 102.

Step 1108: cutting the first base substrate 105 formed with the microcavity 101 into small pieces to form the microfluidic substrate 204A. The area of the microfluidic substrate 204A is about 17×17 mm², and the area of the region where the multiple microcavities 101 are located is about 15×15 mm². There is a distance between the region where the microcavities 101 are located and the edge of the microfluidic substrate 204, which can avoid damage to the microcavity region during the cutting process and leave enough space for subsequent encapsulation.

Step 1109: preparing the second substrate 202.

Cutting the large-sized substrate to obtain the second substrate 202. In an example, the size of the second substrate 202 is 40×42 mm². The second substrate 202 comprise an inlet hole 2021 and an outlet hole 2022. The shape of the inlet hole 2021 and the outlet hole 2022 is circular, and the diameter of the inlet hole 2021 and the outlet hole 2022 is about 0.5-1.5 mm. The second substrate 202 may be made of various suitable materials, such as glass.

Step 1110: preparing the first substrate 201.

Cutting the large-sized substrate to obtain the first substrate 201. The first substrate 201 may be made of various suitable materials, such as glass.

Step 1111: fixing the microfluidic substrate 204A. Placing the microfluidic substrate 204A on the first substrate 201, and fixing the four corners of the microfluidic substrate 204A with UV glue after aligning the positioning marks on the first substrate 201, then placing the sealing frame 203 on the first substrate 201, the sealing frame 203 surrounding the periphery of the microfluidic substrate 204A. The sealing frame 203 may be an elastic sealing frame. In an example, the elastic sealing frame 203 and the first substrate 201 may be subjected to plasma activation treatment first, and then the sealing frame 203 after treatment is placed on the first substrate 201, so that the sealing frame 203 surrounds the peripheral of the microfluidic substrate 204A. The thickness of the sealing frame 203 is about 0.1-0.3 mm thicker than the thickness of the microfluidic substrate 204A, that is, the height of the sealing frame 203 is about 0.1-0.3 mm higher than the height of the microfluidic substrate 204A, taking the first substrate 201 as a reference plane. The sealing frame 203 is configured to keep an appropriate distance between the first substrate 201 and the second substrate 202 and keep the microfluidic chip 200A in a sealed state. In some embodiments, the sealing frame 203 is made of silicone material and has a certain shape by die-cutting. The sealing frame 203 comprises a first side 2031 and a second side 2032 arranged along a first direction D1 and opposite to each other, and a third side 2033 and a four side 2034 arranged along a second direction D2 different from the first direction D1 and opposite to each other. The first side 2031 and the second side 2032 are arc-shaped. In an example, the shape of the first side 2031 and the second side 2032 of the sealing frame 203 is a circular arc. Such arc or circular arc design is beneficial to promote the flow and gathering together of the sample solution in the microfluidic chip 200A, and can avoid residual air bubbles in the microfluidic chip 200A during the sample injection process.

Step 1112: injecting sample. Scraping the mixed sample solution across the surface of the microcavity 101 with a glass scraper to allow the sample solution to flow into the microcavity 101, then using a pipette gun to drop a few drops of fluorinated oil above the microcavity 101 region, the fluorinated oil can be FC-40 or other mineral oils, after the fluorinated oil is flattened, covering the second substrate 202.

Step 1113: sealing treatment. The first substrate 201 and the sealing frame 203 treated by plasma can be packaged, or a certain amount of UV glue may be injected around the sealing frame 203 through a syringe, and leakage can be prevented by sealing the peripheral region. Use a pipette gun to inject fluorinated oil or mineral oil from the inlet hole 2021 of the second substrate 202. After the internal cavities of the microfluidic chip 200A are filled with the fluorinated oil or mineral oil, the inlet hole 2021 and the outlet hole 2022 of the second substrate 202 are sealed with a parafilm or UV glue.

The preparation method of the microfluidic chip 200B illustrated in FIG. 9B is basically the same as the preparation method of the microfluidic chip 200A illustrated in FIG. 9A, with only differences in some steps. For the same method steps, reference may be made to the description of the preparation method of the microfluidic chip 200A, and only the differences of the preparation method of the microfluidic chip 200B will be introduced below.

The same method steps and preparing sequence as steps 1101-1108 are used to prepare the microfluidic substrate 204B, and the same method step as step 1109 is used to prepare the second substrate 202.

The preparation method of the first substrate 201 of the microfluidic chip 200B is different from the preparation method of the first substrate 201 of the microfluidic chip 200A. The preparation method of the first substrate 201 of the microfluidic chip 200B is generally as follows.

Step A: providing a second base substrate 2011. The second base substrate 2011 may be made of any suitable material, and in an example, the second substrate 2011 is made of glass.

Step B: forming a conductive film on the second base substrate 2011 at about 240° C. In an example, a molybdenum (Mo) layer with a thickness of 200 Å, an aluminum neodymium (AlNd) layer with a thickness of 3000 Å, and a molybdenum (Mo) layer with a thickness of 800 Å are sequentially deposited on the second base substrate 2011 to form a conductive film. The conductive film is patterned, such as exposed, developed, etched, etc., to form a conductive layer 2015.

Step C: depositing a first insulating layer on the conductive layer 2015 at about 200° C., and patterning the first insulating layer to form a first dielectric layer 2013 covering the conductive layer 2015. In an example, the first dielectric layer 2013 is a SiO₂ layer with a thickness of about 3000 Å.

Step D: patterning the first dielectric layer 2013 to form at least one via 2016 penetrating the first dielectric layer 2013, the at least one via 2016 exposing a portion of the conductive layer 2015.

Step E: depositing a conductive film on a side of the first dielectric layer 2013 away from the second base substrate 2011, and then performing processes such as exposure, development, etching, and stripping on the conductive film to form a patterned heating electrode 2012. In an example, the material of the heating electrode 2012 is ITO.

Step F: depositing a second insulating layer on a side of the heating electrode 2012 away from the second base substrate 2011, and patterning the second insulating layer to form a second dielectric layer 2014 at least partially covering the heating electrode 2012. In an example, the second dielectric layer 2014 comprises a SiO₂ layer with a thickness of about 1000 Å and a SiNₓ layer with a thickness of about 2000 Å stacked in sequence.

Then, the same preparation method and operation sequence as steps 1111-1113 are used to sequentially realize the fixation, sample injection treatment and sealing treatment of the microfluidic substrate 204B to form the microfluidic chip 200B.

The preparation method of the microfluidic chip 200C illustrated in FIG. 9C is basically the same as the preparation method of the microfluidic chip 200A illustrated in FIG. 9A, with only differences in some steps. For the same method steps, reference may be made to the description of the preparation method of the microfluidic chip 200A, and only the differences of the preparation method of the microfluidic chip 200C will be introduced below.

The same method steps and fabrication sequence as steps 1101-1104 are used to respectively provide the first base substrate 105, form the mark, form the hydrophobic layer 103 and form the mask pattern 205.

Then, after step 1104, another hydrophobic layer and another metal mask pattern are sequentially formed on the surface (i.e., the back surface) of the first base substrate 105 away from the metal mask pattern 205 by marking alignment. The position of another hydrophobic layer completely corresponds to the position of the hydrophobic layer 103, and the position of another metal mask pattern completely corresponds to the position of the metal mask pattern 205. The preparation method of another hydrophobic layer and another metal mask pattern is the same as steps 1103 and 1104.

Step 1105′: immersing the microfluidic substrate 204C formed with the metal mask pattern 205 and another metal mask pattern in an etchant to etch both surfaces of the first base substrate 105. The concentration of hydrogen fluoride (HF) in the etchant is about 40%, and the etching speed is about 3.5 μm/minute. During the etching, the blades are used to continuously stir the etchant, so that the etchant can etch the first base substrate 105 more uniformly. The etching time needs about 60 minutes to form the microcavity 101, which is a through hole. The shape of the opening of the microcavity 101 may be circular, with a diameter of about 50-200 μm, such as 50 μm, 80 μm, 100 μm, 120 μm, 150 μm, 200 μm, etc. The depth of the microcavity 101 is about 300-400 μm, such as 300 μm, 350 μm, 400 μm, and so on. For the specific shape of the microcavity 101, reference may be made to the foregoing descriptions of FIG. 2A and FIG. 2B, and for the sake of brevity, details are not repeated here. Since the through-hole structure of the microcavity 101 has a deep depth, much sample solution can be accommodated, so that more doses of sample solution can be reacted at the same time. In some embodiments, the shape of the second opening 104 of the hydrophobic layer 103 is circular, the shape of the opening of the microcavity 101 is circular, and the diameter of the second opening 104 of the hydrophobic layer 103 is larger than the diameter of the opening of the microcavity 101 by 5-20 μm.

Step 1106′: removing the metal mask pattern 205 and another metal mask pattern after the through-hole microcavity 101 is etched.

Step 1107′: preparing the hydrophilic layer 102 by the same method as step 1107. Since the microcavity 101 is a through hole, the hydrophilic layer 102 is only located on the sidewall of each microcavity 101.

Then, the preparation of the microfluidic chip 200C is completed using substantially the same method steps and manufacturing sequence as steps 1108-1113. However, in step 1111, when using UV glue to fix the four corners of the microfluidic substrate 204C, the UV glue may be doped with 100 μm spacers. The UV glue doped with spacers can not only play a role of fixing, but also provide better support.

The preparation method of the microfluidic chip 200D illustrated in FIG. 9D is basically the same as the preparation method of the microfluidic chip 200A illustrated in FIG. 9A, with only differences in some steps. For the same method steps, reference may be made to the description of the preparation method of the microfluidic chip 200A, and only the differences of the preparation method of the microfluidic chip 200D will be introduced below.

The microfluidic substrate 204D is prepared using the same method steps and preparation sequence as the microfluidic substrate 204C of the microfluidic chip 200C, and the second substrate 202 is prepared using the same method steps as step 1109.

The first substrate 201 of the microfluidic chip 200D is prepared by using steps A-F of the method for preparing the first substrate 201 of the microfluidic chip 200B.

Then, the same preparation method and operation sequence as steps 1111-1113 are used to sequentially realize the fixation, sample injection treatment and sealing treatment of the microfluidic substrate 204D to form the microfluidic chip 200D.

The preparation method of the microfluidic chip 200E illustrated in FIG. 9E is roughly as follows.

The steps of preparing the microfluidic substrate 204E.

Step I: providing a first base substrate 105 and cleaning it. The first base substrate 105 may be made of any suitable material, and in an example, the first base substrate 105 is made of glass. The first base substrate 105 may have any suitable thickness, and in an example, the thickness of the first base substrate 105 is 300-700 μm.

Step II: coating a shielding film on the first base substrate 105, and patterning the shielding film to form a shielding layer 107 defining a first opening 108. In an example, the specific steps of forming the shielding layer 107 may comprise: spin-coating the shielding film on the first base substrate 105 under the condition of a pressure of 30 KPa, the speed of spin-coating is about 380 rpm, and the time for spin-coating is about 7 seconds. Then, pre-curing the spin-coated shielding film at 90° C. for 120 seconds. Next, the shielding film is exposed, developed, and etched through a mask, and the developing time is about 75 seconds. Finally, the etched shielding film is post-cured at 230° C. for about 20 minutes to form the shielding layer 107 defining the first opening 108. In an example, the material forming the shielding layer 107 comprises chromium, chromium oxide, and black resin.

Step III: coating a defining film on a side of the shielding layer 107 away from the first base substrate 105, and patterning the defining film to form a defining layer 106 defining a plurality of microcavities 101. Each microcavity 101 may be a through hole or a blind hole. In an example, the process of forming the defining layer 106 is described as follows: first, under the pressure of 30 Kpa, the surface of the shielding layer 107 away from the first base substrate 105 is spin-coated with the optical glue at a speed of 300 rpm, and the spin-coating time is about 10 seconds, and then at a temperature of 90° C., the optical glue is cured for 120 seconds. Repeat the above process twice to obtain a defining film. Next, the defining film is exposed through a mask, and then a developer is used to develop the exposed defining film for 100 seconds, and then the defining film is etched. At a temperature of 230° C., the etched defining film is cured for 30 minutes, and finally a defining layer 106 defining a plurality of microcavities 101 is obtained. The material of the defining layer 106 comprises photoresist. The orthographic projection of each first opening 108 of the shielding layer 107 on the first base substrate 105 at least partially overlaps the orthographic projection of a corresponding microcavity 101 of the defining layer 106 on the first base substrate 105, and the orthographic projection of the shielding layer 107 on the first base substrate 105 at least partially overlaps the orthographic projection of the defining layer 106 on the first base substrate 105.

Step IV: at 200° C., depositing an insulating layer on the surface of the defining layer 106 away from the first base substrate 105, and performing exposure, development, and etching on the insulating layer to form a patterned layer. The patterned layer is treated with 0.4% KOH solution for about 15 minutes to carry out hydrophilic modification on the patterned layer, thereby forming a hydrophilic layer 102, and the hydrophilic layer 102 is only located inside the microcavity 101. For example, when the microcavity 101 is a blind hole, the hydrophilic layer 102 covers the sidewall and bottom of the microcavity 101. For another example, when the microcavity 101 is a through hole, the hydrophilic layer 102 covers the sidewall of the microcavity 101. In an example, the hydrophilic layer 102 is a SiO₂ layer with a thickness of about 3000 Å.

Step V: depositing an insulating layer on the surface of the defining layer 106 away from the first base substrate 105, and performing exposure, development, and etching on the insulating layer to form a hydrophobic layer 103. In an example, the process of forming the hydrophobic layer 103 is as follows: depositing a SiNₓ layer with a thickness of about 1000 Å on the surface of the defining layer 106 away from the first base substrate 105, and performing exposure, development, and etching on the SiNₓ layer to form a hydrophobic layer 103 comprising a plurality of second openings 104. The orthographic projection of each microcavity 101 on the first base substrate 105 falls within the orthographic projection of a second opening 104 corresponding to the microcavity 101 on the first base substrate 105. In some embodiments, the shape of the second opening 104 of the hydrophobic layer 103 is circular, the shape of the top opening of the microcavity 101 is circular, and the diameter of the second opening 104 of the hydrophobic layer 103 is 5-20 jam larger than the diameter of the top opening of the microcavity 101.

Step VI: cutting the first base substrate 105 formed with the microcavities 101 into small pieces to form a microfluidic substrate 204E. The area of the microfluidic substrate 204E is about 17×17 mm², and the area of the region where the multiple microcavities 101 are located is about 15×15 mm².

Then, the same preparation method and operation sequence as steps 1109-1113 are used to form the microfluidic chip 200E.

The manufacturing method of the microfluidic chip 200F illustrated in FIG. 9F is roughly as follows.

The microfluidic substrate 204F is prepared in the same way as steps I-VI of the microfluidic chip 200E.

The second substrate 202 is prepared by the same method as step 1109.

The first substrate 201 of the microfluidic chip 200F is prepared by adopting the method steps A-F of preparing the first substrate 201 of the microfluidic chip 200B.

Then, the same preparation method and operation sequence as steps 1111-1113 are used to sequentially complete the fixation, sample injection treatment and sealing treatment of the microfluidic substrate 204F to form the microfluidic chip 200F.

For other technical effects of the preparation method of the microfluidic chip, reference may be made to the previous description of the technical effects of the microfluidic substrate and the microfluidic chip. For the sake of brevity, details are not repeated here.

According to another aspect of the present disclosure, a method for using a microfluidic chip is provided, and the microfluidic chip may be the microfluidic chip described in any of the foregoing embodiments. FIG. 12 illustrates a flowchart of the method 1200, and the method 1200 comprises the following steps.

Step S1201: adding the sample solution into multiple microcavities of the microfluidic chip.

Step S1202: heating the microfluidic chip to make the sample solutions in the multiple microcavities react.

Step S1203: detecting an optical signal emitted by the reacted sample solution in the multiple microcavities with an optical device.

When the microfluidic chip is the microfluidic chip illustrated in FIG. 9A, FIG. 9C, or FIG. 9E, that is, the first substrate 201 comprises the second base substrate 2011 but does not comprise the heating electrode, the step of heating the microfluidic chip in step S1202 may comprise: placing the sealed microfluidic chip in a flat thermal cycler.

When the microfluidic chip is the microfluidic chip illustrated in FIG. 9B, FIG. 9D, or FIG. 9F, that is, the first substrate 201 comprises the second base substrate 2011 and the heating electrode 2012, the step of heating the microfluidic chip in step S1202 may comprise: applying an electric signal to the microfluidic chip to drive the heating electrodes 2012 to heat the multiple microcavities 101, and detecting the temperature of the area where the multiple microcavities 101 are located with a temperature sensor to adjust the current flowing through the heating electrodes 2012 in real time. By integrating the heating electrode 2012 in the first substrate 201, the heating of the microcavity 101 can be realized without external heating equipment and the temperature of the microcavity 101 can be controlled in real time. Therefore, the microfluidic chip comprising the first substrate 201 has higher integration and more precise temperature control, and improves the stability of the microfluidic chip during the heating process.

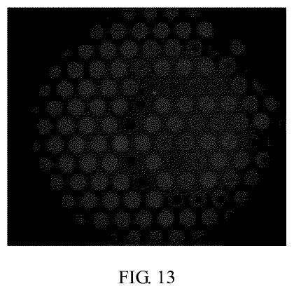

FIG. 13 illustrates a fluorescent image of a microfluidic chip provided according to an embodiment of the present disclosure, and each dot in the image represents a microcavity 101 comprising positive cells. After the sample solution in the microfluidic chip completes the PCR reaction, the sample solution comprising positive cells in the microcavity will emit fluorescence under irradiation of excitation light, while the sample solution comprising negative cells in the microcavity will not emit fluorescence under irradiation of excitation light, therefore, the light and dark quantities of the microcavity can be observed by placing the microfluidic chip under the observation lens. It can be seen from FIG. 13 that the difference between the color of microcavities 101 and the color of the surrounding region of microcavities 101 is huge, and the contrast is apparent. The microcavities 101 present a relatively bright color, while the surrounding region presents a black color. Therefore, the microfluidic chip provided by the embodiments of the present disclosure can provide high resolution and clarity for the fluorescence detection of the sample solution, so that the fluorescence signal emitted by the sample solution in the microcavity 101 can be accurately identified by the detector. Therefore, the reaction signal can be read more sensitively and accurately, the fluorescence detection accuracy of the sample solution can be improved, and image data support can be provided for the data analysis of the subsequent nucleic acid amplification reaction.

It will be understood that although the terms first, second, third, etc. may be used herein to describe various elements, components, regions, layers and/or portions, these elements, components, regions, layers and/or portions should not be limited by these terms. These terms are only used to distinguish an element, component, region, layer or portion from another region, layer or portion. Thus, a first element, component, region, layer or portion discussed above could be termed a second element, component, region, layer or portion without departing from the teachings of the present disclosure.

Spatially relative terms such as “row”, “column”, “below”, “above”, “left”, “right”, etc. may be used herein for ease of description to describe factors such as the relationship of an element or feature to another element or feature(s) illustrated in the figures. It will be understood that these spatially relative terms are intended to encompass different orientations of the device in use or operation in addition to the orientation depicted in the figures. For example, if the device in the figures is turned over, elements described as “below” other elements or features would then be oriented “above” other elements or features. Thus, the exemplary term “below” can encompass both an orientation of above and below. The device may be otherwise oriented (rotated 90 degrees or at other orientations) and the spatially relative descriptors used herein are interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to limit the present disclosure. As used herein, the singular forms “a,” “an,” and “the” are intended to comprise the plural forms as well, unless the context clearly dictates otherwise. It will be further understood that the terms “comprise” and/or “include” when used in this specification designate the presence of stated features, integers, steps, operations, elements and/or parts, but do not exclude the presence or addition of one or more other features, integers, steps, operations, elements, components and/or groups thereof. As used herein, the term “and/or” comprises any and all combinations of one or more of the associated listed items. In the description of this specification, description with reference to the terms “an embodiment,” “another embodiment,” etc. means that a particular feature, structure, material, or characteristic described in connection with the embodiment is included in at least one embodiment of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples as well as the features of the different embodiments or examples described in this specification without conflicting each other.

It will be understood that when an element or layer is referred to as being “on”, “connected to”, “coupled to”, or “adjacent to” another element or layer, it may be directly on, directly connected to, directly coupled to, or directly adjacent to another element or layer, or intervening elements or layers may be present. In contrast, when an element is referred to as being “directly on”, “directly connected to”, “directly coupled to”, “directly adjacent to” another element or layer, with no intervening elements or layers present. However, in no case should “on” or “directly on” be interpreted as requiring a layer to completely cover the layer below.

Embodiments of the disclosure are described herein with reference to schematic illustrations (and intermediate structures) of idealized embodiments of the disclosure. As such, variations to the shapes of the illustrations are to be expected, e.g., as a result of manufacturing techniques and/or tolerances. Accordingly, embodiments of the present disclosure should not be construed as limited to the particular shapes of the regions illustrated herein, but are to comprise deviations in shapes due, for example, to manufacturing. Thus, the regions illustrated in the figures are schematic in nature and their shapes are not intended to illustrate the actual shape of a region of a device and are not intended to limit the scope of the present disclosure.

Unless otherwise defined, all terms (comprising technical and scientific terms) used herein have the same meaning as commonly understood by one of ordinary skill in the art to which this disclosure belongs. It will be further understood that terms such as those defined in commonly used dictionaries should be construed to have meanings consistent with their meanings in the relevant art and/or the context of this specification, and will not be idealized or overly interpreted in a formal sense, unless expressly defined as such herein.

As will be appreciated by those skilled in the art, although the steps of the methods of the present disclosure are depicted in a particular order in the figures, this does not require or imply that the steps must be performed in that particular order, unless the context clearly dictates otherwise. Additionally or alternatively, multiple steps may be combined into one step for execution, and/or one step may be decomposed into multiple steps for execution. Furthermore, other method steps may be inserted between the steps. The inserted steps may represent such as improvements of a method described herein, or may be unrelated to the method. Also, a given step may not be fully complete before the next step starts.

The above descriptions are merely specific embodiments of the present disclosure, but the protection scope of the present disclosure is not limited thereto. Any changes or substitutions that those skilled in the art can easily think of within the technical scope disclosed by the present disclosure, should be comprised within the protection scope of the present disclosure. Therefore, the protection scope of the present disclosure should be based on the protection scope of the claims.

1. A microfluidic substrate comprising a substrate, the substrate comprising a plurality of microcavity regions arranged in an array, wherein each of the plurality of microcavity regions comprises a first portion and a second portion that are stacked, a depth of the first portion is x, the first portion comprises a top opening with a circular shape and a diameter D, and a relationship between the diameter D of the top opening and the depth x is approximately D=2x+y, where a range of x is from 20 microns to 400 microns, and a range of y is from 5 microns to 30 microns.

2. The microfluidic substrate according to claim 1, wherein the first portion is a blind hole and the first portion does not penetrate the second portion, and the first portion constitutes a microcavity of the microfluidic substrate.

3. The microfluidic substrate according to claim 2, wherein the substrate further comprises a third portion which is between any adjacent two microcavity regions in the plurality of microcavity regions and not etched,
wherein in each microcavity region, a portion of the substrate which is etched constitutes the first portion, a portion of the substrate which is not etched constitutes the second portion, an orthographic projection of the first portion on the microfluidic substrate overlaps an orthographic projection of the second portion on the microfluidic substrate, and
wherein the second portion and the third portion are integral.

4. The microfluidic substrate according to claim 2, wherein the range of x is from 20 microns to 100 microns.

5. The microfluidic substrate according to claim 2, wherein a shape of the first portion is a curved surface body, and the first portion comprises the top opening, a bottom, and a sidewall connecting the top opening and the bottom, the bottom of the first portion is circular in shape and has a diameter of about x microns.

6. The microfluidic substrate according to claim 5, wherein a tangent plane at each of at least some points on the sidewall is at a non-perpendicular angle to a reference plane where the microfluidic substrate is located.

7. The microfluidic substrate according to claim 1, wherein the second portion comprises a bottom opening on a side away from the first portion, the first portion penetrates the second portion to form a through hole, and the through hole constitutes a microcavity of the microfluidic substrate.

8. The microfluidic substrate according to claim 7, wherein a depth of the second portion is x and a shape of the bottom opening of the second portion is circular, a relationship between a diameter D of the bottom opening and the depth x is approximately D=2x+y.

9. The microfluidic substrate according to claim 7, wherein the first portion and the second portion have a same shape and are axisymmetric about a symmetry axis, the symmetry axis is parallel to a reference plane where the microfluidic substrate is located.

10. (canceled)

11. (canceled)

12. The microfluidic substrate according to claim 1, further comprising:
a shielding layer,
wherein the substrate comprises a first base substrate and a defining layer on the first base substrate, the defining layer comprises a plurality of first portions and a plurality of second portions,
wherein the shielding layer comprises a plurality of first openings, the plurality of first openings correspond to the plurality of microcavity regions one by one, and an orthographic projection of each of the plurality of microcavity regions on the microfluidic substrate at least partially overlaps an orthographic projection of a first opening corresponding to the microcavity region on the microfluidic substrate, and
wherein an orthographic projection of the shielding layer on the microfluidic substrate at least partially overlaps an orthographic projection of the defining layer on the microfluidic substrate.

13. (canceled)

14. The microfluidic substrate according to claim 1, further comprising:
a spacing region between any two adjacent microcavity regions in the plurality of microcavity regions;
a hydrophobic layer disposed within the spacing region; and
a hydrophilic layer arranged at least in the plurality of microcavity regions,
wherein the hydrophobic layer comprises a plurality of second openings, the plurality of microcavity regions correspond to the plurality of second openings one by one, and an orthographic projection of each microcavity region on the microfluidic substrate falls within an orthographic projection of a second opening corresponding to the microcavity region on the microfluidic substrate,
wherein a shape of the second opening is circular, and a diameter of the second opening is larger than the diameter of the top opening, and
wherein an orthographic projection of a portion of the hydrophilic layer arranged in each microcavity region on the microfluidic substrate falls within the orthographic projection of the second opening corresponding to the microcavity region on the microfluidic substrate.

15. (canceled)

16. (canceled)

17. A microfluidic chip comprising:
a first substrate;
a second substrate opposite to the first substrate;
the microfluidic substrate according to claim 1, the microfluidic substrate being between the first substrate and the second substrate; and
a sealing frame between the first substrate and the second substrate,
wherein an orthographic projection of the microfluidic substrate on the first substrate falls within an orthographic projection of the sealing frame on the first substrate.

18. The microfluidic chip according to claim 17, wherein the sealing frame comprises a first side and a second side arranged along a first direction and opposite to each other, and a third side and a fourth side arranged along a second direction different from the first direction and opposite to each other, a shape of the first side and the second side is an arc.

19. The microfluidic chip according to claim 18, wherein the microfluidic substrate comprises a first edge and a second edge arranged along the second direction and opposite to each other, a distance between an orthographic projection of the third side of the sealing frame on the first substrate and an orthographic projection of the first edge of the microfluidic substrate on the first substrate is 2 mm to 6 mm, and a distance between an orthographic projection of the fourth side of the sealing frame on the first substrate and an orthographic projection of the second edge of the microfluidic substrate on the first substrate is 2 mm to 6 mm.

20. (canceled)

21. The microfluidic chip according to claim 17, wherein the second substrate comprises an inlet hole and an outlet hole, and orthographic projections of the inlet hole and the outlet hole on the first substrate fall within the orthographic projection of the sealing frame on the first substrate.

22. (canceled)

23. The microfluidic chip according to claim 17, wherein the first substrate comprises:
a second base substrate; and
a heating electrode between the second base substrate and the microfluidic substrate,
wherein an orthographic projection of the plurality of microcavity regions of the microfluidic substrate on the second base substrate falls within an orthographic projection of the heating electrode on the second base substrate.

24. The microfluidic chip according to claim 23, wherein an orthographic projection of the sealing frame on the second base substrate falls within the orthographic projection of the heating electrode on the second base substrate.

25. The microfluidic chip according to claim 23, wherein the first substrate further comprises:
a first dielectric layer between the second base substrate and the heating electrode;
a second dielectric layer between the heating electrode and the microfluidic substrate; and
a conductive layer between the second base substrate and the first dielectric layer,
wherein the conductive layer is electrically connected to the heating electrode through a via in the first dielectric layer.

26. (canceled)

27. (canceled)

28. (canceled)

29. (canceled)

30. (canceled)

31. A method of using the microfluidic chip according to claim 17, comprising:
adding a sample solution into a plurality of microcavities of the microfluidic chip;
heating the microfluidic chip to react the sample solution in the plurality of microcavities; and
detecting an optical signal emitted by the reacted sample solution in the plurality of microcavities with an optical device.

32. (canceled)

33. The method according to claim 31,
wherein the first substrate comprises a second base substrate, and wherein the heating the microfluidic chip, comprises: placing the microfluidic chip in a flat thermal cycler; or,
wherein the first substrate comprises a second base substrate and a heating electrode between the second base substrate and the microfluidic substrate, and an orthographic projection of the plurality of microcavities of the microfluidic substrate on the second base substrate falls within an orthographic projection of the heating electrode on the second base substrate, and
wherein the heating the microfluidic chip, comprises applying an electrical signal to the microfluidic chip to drive the heating electrode to heat the plurality of microcavities, and detecting a temperature of the plurality of microcavity regions by a temperature sensor to adjust a current flowing through the heating electrode in real time.
